(19) United States
(12) Patent Application Publication
(10) Pub. No.: US 2008/0213922 A1
(43) Pub. Date: Sep. 4, 2008

(54) Method And System For Identification Of Antigen

(76) Inventor: Francina C. Chahal, Winnipeg (CA)

Correspondence Address: BERESKIN AND PARR, 40 KING STREET WEST, BOX 401, TORONTO, ON, M5H 3Y2, CA

(21) Appl. No.: 11/909,939

(22) PCT Filed: Apr. 4, 2006
(86) PCT No.: PCT/CA06/00514

§ 371 Date: Jan. 9, 2008

Related U.S. Application Data
(60) Provisional application No. 60/667,687, filed on Apr. 4, 2005.
(60) Provisional application No. 60/751,967, filed on Dec. 21, 2005.

(51) Int. Cl. G01N 33/531

(52) U.S. Cl. 436/543

(57) ABSTRACT

There is provided a method for the identification of antigens recognized by a given antibody. In particular the method provides for characterization of the epitope recognized by the antibody and for purification based on the physico-chemical properties of the antigen. The characterization facilitates subsequent analysis of the antigen for identification purposes.

### CROSS-REFERENCE TO RELATED APPLICATION

This application claims priority on US provisional application No. 60/667,687 entitled “METHOD AND SYSTEM FOR MASS SPECTROMETRY IDENTIFICATION OF ANTIGEN” and filed Apr. 4, 2005 and on US provisional application No. 60/751,967 entitled “METHOD AND SYSTEM FOR MASS SPECTROMETRY IDENTIFICATION OF ANTIGEN” and filed on Dec. 21, 2005.

### FIELD OF THE INVENTION

This invention relates to the field of antigen identification. More specifically the invention relates to the identification of protein antigens using multi-dimension separation and mass spectrometry.

### BACKGROUND OF THE INVENTION

Identification of antigens that are differentially expressed or expressed in diseased tissues such as tumors is of great importance for understanding fundamental biological processes such as development and disease progression. Furthermore, antigen identification can also provide information on potential drug targets.

Tumor antigens are generally membrane proteins possessing one or more transmembrane domains. In some cases, they serve as effective signal transducers and therefore tend to demonstrate changes in conformation (Atassi and Smith, 1978. Immunochemistry, 15:609-610). Fractionation and purification of tumor antigens leading to their identification, has been a challenge for a long time. Two-dimensional gel approaches have been used extensively to fractionate proteins on the basis of isoelectric points/molecular weight (O' Farrel, P. H. 1975. J. Biol. Chem. Vol: 250:4007; Gorg, A., Obermaier, C., Soguth, G., Harder, A., Schiebe, B., Wildgruber, R. and Weiss, W. 2000: Electrophoresis Vol: 21 (6): 1037; Santoni et al. Electrophoresis. 2000October; 21(16):3329-44).

Conventional two-dimensional liquid phase analysis (2D-LC) comprise separation on a strong cation exchange column (SCX), followed by separation on the basis of hydrophobicity, while other systems such as the ProteomeLab PF-2D, use chromatofocusing as the first dimension (Xiang R. et al 2004. J Proteome Res. November-December; 3(6): 1278-83; Fujii K, Nakano T, Hike H, Usui F, Bando Y, Tojo H, Nishimura T. 2004. J Chromatogr A. November 19; 1057(1-2): 107-13).

While these techniques complement each other and hence have proven successful in some cases, they do not satisfy the conditions that promote the effective fractionation and identification of certain proteins such as membrane antigens. A combination of RP-HPLC and SDS-PAGE has also been suggested for membrane protein separations (O'Neil K A, Miller F R, Barder T J, Lubman D M. 2003. Proteomics. July; 3(7): 1256-69). However, limitations due to hydrophobicity, higher molecular weights, posttranslational modifications and solubilities, generally observed in a gel-based approach still persist with these procedures. Furthermore, only small amounts of the protein of interest can be separated.

There is therefore a need for better antigen identification methods.

### SUMMARY OF THE INVENTION

In a broad aspect of the invention there is provided a method for the identification of one or more antigens in samples such as biological samples. An antibody is provided for which the cognate antigen is unknown. Samples are first screened to identify those containing the antigen and the antigen is then characterized to be identified.

In an embodiment the method comprises a pre-purification step based on the antigen's epitope properties. The pre-purification advantageously facilitate the subsequent analysis of the antigen.

In one embodiment the antigen is a protein and there is accordingly provided a multi-dimension protein separation method that advantageously improves protein identification. In one embodiment the method comprises a pre-purification step that precedes 2-dimensional protein separation and identification by mass spectrometry. The pre-purification allows enrichment of protein fractions in one or more target proteins thereby enabling the optimization of subsequent (downstream) separation/identification steps. The method is particularly useful to identify proteins in sub-cellular compartments, such as membrane proteins, hydrophobic proteins with higher molecular weights, posttranslational modifications and limited solubilities and proteins that are differentially expressed.

In another aspect of the invention there is provided a multi-dimension protein separation method in which protein are enzymatically digested at different stages of the purification/separation process to generate peptide maps enabling the optimization of subsequent (downstream) separation/identification steps.

In yet another aspect there is also provided a system for identifying an antigen comprising pre-purification means for providing a fraction enriched in the antigen separating means for separating the antigen from other components in the fraction; and an analysis means for identifying the antigen.

In another embodiment there is also provided a method for determining an antigen identification protocol the method comprising providing an antibody for which it is desired to identify the corresponding antigen characterizing an epitope of the antibody on the antigen; and selecting the protocol based on one or more properties of the epitope.

In the present application by antigen it is meant any biological macromolecule capable of eliciting an immune response that results in the production of antibodies. Such antigens include but are not limited to proteins, glycoproteins, lipids, glycolipids, carbohydrates and nucleic acids. In a preferred embodiment the antigen is present in an individual afflicted by a disease. In a more preferred embodiment the antigen is present in diseased cells but not on normal cells.

In the present application by epitope properties it is meant any characteristic of the epitope that can be exploited for the purification of the antigen including but not limited to the nature of the epitope (carbohydrate, amino acids, etc.), hydrophobicity, degree of glycosylation, degree of accessibility by the antibody (masking), charge, and the like.

### BRIEF DESCRIPTION OF THE DRAWINGS

Further features and advantages of the present invention will become apparent from the following detailed description, taken in combination with the appended drawings, in which:

FIG. 1 is a schematic diagram of some embodiments of the invention;

FIG. 2 is an elution profile of ProteomeLab™ PF-2D fractionation of SKBR-3 and HepG2 cell extract;

FIG. 3 is an elution profile of ProteomeLab™ PF-2D fractionation of SKBR-3 and HepG2 cell extract showing the peaks eluting at around 27, 28 and 29 minutes;

FIG. 4 is a dot blot of ProteomeLab™ PF-2D fractions from HepG2 and SKBR-3; and

FIG. 5 is a Western blot of PF-2D fractions from SKBR-3 and HepG2.

FIG. 6 shows the fractionation profiles of two positive cells lines, HepG2 and MCF-7 and of two negative cell lines, Panc-1 and C-33A on a PF-2D system. This figure represents a chromatographic file from 10 to 25 minutes.

FIG. 7 shows TOF-MS scans of peptides obtained from HepG2 cell line, to detect the presence of all peptide ions in the sample. The figure shows the results from fifty-three scans at 1200-1400V in the range of 100-1200 amu on a static nanospray

FIG. 8 shows TOF-MS scans of peptides obtained from Panc-1 cell line, to detect the presence of all peptide ions in the sample (Thirty scans at 1200-1400V in the range of 100-1200 amu on a static nanospray).

FIG. 9 shows TOF-MS scans of peptides obtained from MCF-7 cell line, to detect the presence of all peptide ions in the sample (Twenty-seven scans at 1200-1400V in the range of 100-1200 amu on a static nanospray)

FIG. 10 shows TOF-MS scans of peptides obtained from C-33A cell line, to detect the presence of all peptide ions in the sample (Thirty scans at 1200-1400V in the range of 100-1200 amu on a static nanospray).

FIG. 11 shows the sequence coverage of peptides recovered from mass spectrometry analysis as listed in Table 1. Sequences underlined represent the peptide sequences recovered and bolded sequences show the amino acid sequences where homology was less than 100%.

FIG. 12 shows the peptide mass fingerprinting results for the peptides recovered from VB1-050 Antigen. Protein scores greater than 64 were considered significant.

FIG. 13 shows accession number, mass and score of the MS/MS fragmentation and name of proteins retrieved from protein database searching.

FIG. 14 shows the MS/MS ion fragmentation of the neutral peptide Mr. 1401.54, appearing as a triply charged molecule (466.60000, 3+). The peptide sequence showed 100% homology with a peptide from Glucose Transporter 8.

FIG. 15 shows the MS/MS ion fragmentation of the neutral peptide Mr. 1070.785, appearing as a doubly charged molecule (536.40000, 2+). The peptide sequence showed 100% homology with a peptide from Glucose Transporter 8.

FIG. 16 shows the MS/MS ion fragmentation of the neutral peptide Mr. 1997.9992, appearing as a triply charged molecule (667.098230, 3+). The peptide sequence showed changes in amino acids at positions 7, 10, 12, 13, 14, 15 and 18; compared to the homologous peptide from Glucose Transporter 8.

FIG. 17 shows the MS/MS ion fragmentation of the neutral peptide Mr. 1176.3547, appearing as a doubly charged molecule (589.100000, 2+). The peptide sequence showed changes in amino acids at positions 7, 10, 12, 13, 14 and 15; compared to the homologous peptide from Glucose Transporter 8.

FIG. 18 shows glycan structures recognized by the VB3-011 antibody. Chondroitin sulphate A, also known as Chondroitin-4-sulphate, (due to the presence of the Sulfate molecule at position 4), is a linear molecule of repeating D-galactosamine and glucuronic acid (A). When two such CSA molecules get cross-linked via a 2-6 alpha linkage, the glycan unit now represents one recognized by Heamagglutinin (HA) (B).

FIG. 19 is a schematic representation of HA reagent immobilization for lectin-based purification of antigen.

FIG. 20 shows a SDS-PAGE/Western blot of the proteins obtained from lectin-based purification of proteins from U87MG, U118MG, A375, Panc-1 and Daudi Lectins were Con-A, WGA and HA.

FIG. 21 a SDS-PAGE/Western blot of the pre-purified proteins form A375 and U118MG where the SDS-PAGE was performed with proteins incubated at room temperature for 1 hour.

FIG. 22 shows Western blot profile of the 2D-PAGE obtained from HA-based purification of cell proteins. The blot was probed with the VB3-011 antibody

FIG. 23 shows the complete mapping of the peptides obtained and the sequence coverage of the Scratch molecule, Accession # gi|13775236. The underlined amino acids represent the sequences of amino acids identified from MS analysis.

FIG. 24 shows TOF-MS scans of peptides obtained from A-375 cell line, to detect the presence of all peptide ions in the sample. One hundred scans at 1200-1400V in the range of 100-1200 amu on a static nanospray resulted in the recovery of a significant number of peptides, which were analyzed for protein identification. FIG. 24A represents the TOF-MS scan with all multiply charged peptide ions and FIG. 24B represents the deconvoluted spectrum with singly charged peptide ions.

FIG. 25 shows TOF-MS scans of peptides obtained from U87MG cell line, to detect the presence of all peptide ions in the sample. Three hundred scans at 1200-1400V in the range of 100-1200 amu on a static nanospray resulted in the recovery of a significant number of peptides, which were analyzed for protein identification. FIG. 25A represents the TOF-MS scan with all multiply charged peptide ions and FIG. 25B represents the deconvoluted spectrum with singly charged peptide ions.

FIG. 26 shows TOF-MS scans of peptides obtained from U87MG cell line, to detect the presence of all peptide ions in the sample. Twenty-seven scans at 1200-1400V in the range of 100-1200 amu on a static nanospray resulted in the recovery of a significant number of peptides, which were analyzed for protein identification. FIG. 26A represents the TOF-MS scan with all multiply charged peptide ions and FIG. 26B represents the deconvoluted spectrum with singly charged peptide ions.

FIG. 27 shows the sequence coverage of peptides recovered from mass spectrometry analysis as listed in Table 3. Underlined sequences represent the peptide sequences recovered. The sequences in bold are the sequences with less than 100% homolgogy and the ones in italics represent those that are 100% homologous to Mammalian Scratch peptides.

FIG. 28 shows the peptide mass fingerprinting results for the peptides recovered from VB3-011Ag. Protein scores greater than 77 were considered significant.

FIG. 29 shows accession number, mass and score of the MS/MS fragmentation and name of proteins retrieved from protein database searching.

FIG. 30 shows the MS/MS ion fragmentation of the neutral peptide Mr. 2402.978172, appearing as a triply charged molecule (802.00000, 3+). The peptide sequence show 100% homology with a peptide from Scratch.

FIG. 31 shows the MS/MS ion fragmentation of the neutral peptide Mr. 2134.985448, appearing as a doubly charged molecule (1068.500000, 2+). The flanking regions of the recovered peptide are homologous to the peptide from Scratch; the rest of the sequence is no more than 40% homolous to the peptide from Scratch.

### DETAILED DESCRIPTION OF THE INVENTION

The present invention provides a method for the identification of an antigen recognized by a given antibody. The antibody is preferably obtained from an individual afflicted by a disease so that identification of the cognate antigen can assist in the development of a therapeutic or diagnostic approach such as by providing a drugable target. The antibody can be obtained by various approach such as that described in patent application WO2005121341.

In a first step samples are screened to detect the presence of the antigen. Screening can be achieved by a binding assay using a labeled antibody, by a blotting assay or other assays as would be known to one skilled in the art. It will be appreciated that samples that do not exhibit the antigen can be used as controls in the subsequent steps of antigen identification. Once a sample has been identified as comprising the antigen, the sample is analyzed to provide an identification of the antigen.

In a preferred embodiment the antigen is pre-purified based on the properties of the epitope recognized by the antibody, This pre-purification advantageously improves and facilitate subsequent separation and analysis steps.

Characterization of the antigen comprises separation of the antigen from other components of the sample such as to provide an antigen sufficiently pure to be analyzed by analytical tools such as mass spectrometry, nuclear magnetic resonance, sequencing and the like.

It will be appreciated that while the antigens described herein are proteins, the method and system of the invention can be used for other types of antigens such as carbohydrates, lipids and the like.

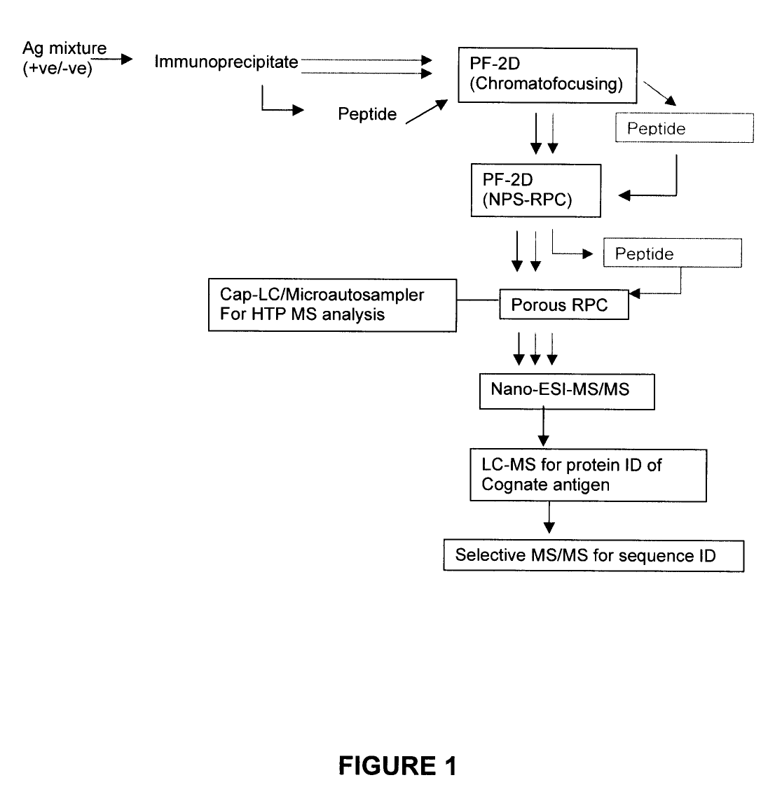

In one aspect of the method for the separation and identification of protein antigens multi-dimension separation and mass spectrometry analysis (see FIG. 1) is used. By multi-dimension separation it is meant protein separation comprising two or more separation steps based on two or more different physico-chemical properties of the proteins.

In one embodiment protein preparations from which it is desired to identify one or more protein antigen using multi-dimension separation are subjected to a pre-purification step to generate protein fractions enriched in the proteins of interest. The enriched fractions are subsequently separated using multi-dimension separation for the purpose of being mapped and/or identified.

The pre-purification step is based on one or more physico-chemical characteristics of the antigen and preferably of the epitope recognized by the antibody. The characteristics may comprise but are not limited to hydrophobicity, molecular weight, charge, affinity and the like. The pre-purification step may employ one or more affinity separation methods. For example, the pre-purification step may comprise immunoprecipitation with an antibody specific for a protein antigen. Other affinity protein purification approaches may also be used. For example in the case where the epitope is mostly carbohydrates, an affinity matrix can be designed to specifically bind the epitope. Other affinity chromatography approaches such as, but not limited to ligand exchange.

The identity of the protein does not need to be known prior to the purification and identification procedure. For example, it is not necessary to know the identity of the protein recognized by the antibody prior to the pre-purification step. In fact the method can be used for identifying a protein (or proteins) recognized by an antibody (or antibodies) raised against unknown antigens. For example, antibodies raised against cancer cell antigens.

The pre-purification step may also comprise the isolation of a sub-cellular structure/(compartment/organelle) to facilitate the purification of proteins localized in that particular sub-cellular structure. In one example, the sub-cellular structure is the cell membrane.

The pre-purification step is followed by one or more protein separation steps that can involve several dimensions. The first dimension separates proteins based on a first physico-chemical property. For example, in some embodiments of the present invention, proteins are separated by chromatofocusing (based on pi of protein using isoelectric focusing) in the first dimension. It will be appreciated that the first dimension may employ any number of separation techniques including, but not limited to, ion exclusion, ion exchange, normal/reversed phase partition, size exclusion, ligand exchange, liquid/gel phase isoelectric focusing, and adsorption chromatography.

In the second dimension, proteins are separated based on a second physico-chemical property different from that used in the first dimension. In one embodiment the second dimension separation is based on the hydrophobic properties of the proteins using, for example, reverse phase (RP) liquid chromatography. In one embodiment, the RP chromatography is performed using non-porous reverse phase (NP RP) chromatography and the liquid chromatography is performed using high pressure liquid chromatography (HPLC). Other examples of protein separations using PI and NP RP can be found in Buchanan et al. Electrophoresis. 2005 January;26(1):248-56; Wang et al. Proteomics. 2004 August;4(8):2476-95 and US patent application No. 20040010126 all references incorporated herein by reference.

The proteins separated by the second phase are then analyzed by mass spectrometry using, for example, liquid chromatography-mass spectrometry (LC-MS), nano-electrospray ionization tandem mass spectrometry (ESI-MS/MS), tandem mass spectrometry (MS/MS) and the like (see for examples Protein Sequencing and Identification Using Tandem Mass Spectrometry, M. Kinter and N. Sherman, John Wiley & Sons D. Desiderio and M. Nibbering eds., 2000; The expanding role of Mass Spectrometry in Biotechnology, G. Siuzdak, MCC Press 2003 and U.S. Pat. No. 6,656,690 all references incorporated herein by reference).

Protein purification and separation at any given step is preferably conducted in the liquid phase in a buffer that is preferably compatible with the next (downstream) separation/identification step. Thus products of one separation step can be fed directly into the next liquid phase separation step therefore facilitating the automation of the process and providing for high throughput processing. It will be appreciated however that the sample may need to be processed to adjust the liquid phase parameters such as pH, ionic strength etc. between the different purification/separation steps so as to adjust the condition to be compatible to the next step. It will also be appreciated that the buffer is preferably compatible with mass spectrometry. Performing the purification and separation in liquid phase also allows the pooling of fractions.

The method of the present invention can be advantageously applied to the identification of membrane proteins. Membrane proteins are difficult to process for separation and identification due in part to their hydrophobicity. Pre-purification of membrane proteins according to the present invention prior to multi-dimensional separation can improve the efficiency of the separation. Thus in one embodiment one or more membrane protein can be pre-purified using, for example, affinity purification such as immunoprecipitation.

The protein fractions obtained from the second dimension can be displayed in a 2-D map with each dimension corresponding to the separation based on one particular physico-chemical characteristic. As will be known to those skilled in the art the map can be generated using specialized software.

It is often desirable to compare the expression of one or more proteins between cells. For example, one or more proteins may be differentially expressed or specifically expressed in certain cell types as for example in diseased tissue. Because of the complexity of protein expression the protein maps resulting from 2D separations of two different cell types can be difficult to compare and can impede the identification of one or more differentially expressed proteins. The method of the present invention advantageously facilitates the comparison of protein profiles between two or more samples (e.g., cancer vs. control cells, undifferentiated vs. differentiated cells, treated vs. untreated cells). The pre-purification step can be designed to enrich the protein fractions with one or more target proteins therefore simplifying the profiles of subsequent steps.

Thus two samples to be compared can be run in parallel. The data obtained from each of the samples is compared to determine differences in protein expression between the samples. The profile for a given cell type may be used as a control (standard) for determining the presence or absence of proteins of interest in unknown samples. The sample can be further characterized by identifying one or more proteins of interest in the expression pattern using mass spectrometry. It will be appreciated that the proteins from different samples may also be run simultaneously. In this case, the proteins from each sample may be separately labeled to allow the protein expression patterns from each sample to be distinguished and displayed.

In another aspect of the invention the proteins are enzymatically digested prior to mass spectrometry analysis for peptide mapping. In one embodiment of the invention enzymatic proteolysis is performed during separation. That is to say, it can be performed either prior to multi-dimension separation, after separation in the first dimension or after separation in the second dimension. Comprehensive peptide mapping at different steps of the purification/separation procedure allows a more precise identification of the protein and therefore a more accurate processing in subsequent separation/identification steps. It will be appreciated that peptide generation at different stages of the process may be performed with or without the pre-purification step. Protein digestion for mass spectrometry is generally described for example in Protein Sequencing and Identification Using Tandem Mass Spectrometry, M. Kinter and N. Sherman, John Wiley & Sons D. Desiderio and M. Nibbering eds., 2000 incorporated herein by reference.

The pre-purification step and the enzymatic digestion of proteins advantageously allow the samples to be further separated prior to the identification by mass spectrometry. For example, the fractions obtained after 2D separation can be injected in a mass spectrometer through a porous RP chromatography. This additional step may enable greater resolution of the fractions and, accordingly, assist in the identification of the proteins.

The method of the present invention may also be used to isolate one or more proteins from a sample comprising said one or more proteins, pre-purifying the one or more proteins, using at least one known property of said one or more proteins, to produce protein fractions enriched with the protein(s) and separating proteins comprised in the protein fractions using 2-Dimensional (2D) liquid chromatography.

The methods of the invention may be used to identify novel proteins and such proteins are encompassed within the scope of the invention.

In another aspect of the invention there is also provided a method for determining an antigen identification protocol which is based on the properties of the antigen. In a first step the nature of the epitope (N-/O-glycosylated or a peptide) is determined and it is also determine whether the antigen is “blottable” or “non-blottable” (whether or not it can be detected by gel-based assays). This enables the selection of appropriate subsequent steps for the purification and identification of the antigen.

For blottable antigens the separation can be achieved by 2D-PAGE without recourse to the pre-purification step. For non-blottable antigens, if the inability to “blot” is due to glycosylation of the antigen that masks the epitope, then the antigen (or the membrane comprising the antigen) can be deglycosylated prior to performing the separation.

In the case where the antigen is non-blottable and the glycan is part of the epitope, a prepurification step using lectin based affinity chromatography can be used followed by separation and identification.

In the case where the antigen is non-blottable as a result of extreme hydrophobicity, an immunoprecipitation pre-purification followed by separation, preferably using PF-2D, can be used prior to identification.

In yet another aspect of the invention there is also provided a system for identifying an antigen the system comprising pre-purification means for fractions enriched with the antigen, separating means separation of the antigen from other components of the sample and analysis means to identify the antigen. In one example the separating means for multi-dimension protein separation/identification comprises a first separating means for separating protein fractions received from the pre-purification means based on a first physico-chemical property, a second separating means for separating protein fractions received from the first separating means based on a second physico-chemical property and an analysis means for identifying proteins collected from the second separation means.

The system may also comprise a first protein fraction collecting device for receiving pre-purified protein fractions, a second fraction collecting device for receiving separated fractions from the first separating means, and a third fraction collecting device for receiving separated fractions from the second separating means. The system optionally comprises a third separation means for separating the fractions collected from the second separation apparatus and introducing the fractions in the analysis means. The system may also comprise a processor for analyzing and displaying the fractionation profiles and protein/peptide maps. It will be appreciated that the different part of the system can be coupled to provide for automatic collection/injection of the protein fractions. For example fractions may automatically be collected in multiwell plates, such as 96 wells plates, that can be autosampled using an autosampler. Such standardized collecting/sampling arrangements can facilitate the direct comparison of protein fractions from two different samples.

In one non-limiting example the system may comprise an immunoprecipitation kit, a 2-dimensional protein separation apparatus such as the PF-2D™ system comprising a chromatofocusing apparatus in the first dimension and a reverse phase non-porous HPLC in the second dimension and a mass spectrometer. Optionally the system comprises a porous reverse phase apparatus in line with the reverse phase non-porous HPLC. The fractions may be injected in the porous reverse phase separation apparatus using a capillary LC equipped with a microautosampler for high throughput analysis.

### EXAMPLES

### Example 1

### Antigen Enrichment, PF-2D Fractionation and Mass Analysis

In one embodiment cells positive for one or more antigens and cells that do not express the antigen are provided. In one example cell protein preparation from HER-2-positive and HER-2-negative cell lines are submitted to immunoprecipitation (see below for details) with the anti-HER-2 antibody.

The purified fraction is equilibrated with CF-start (CF: chromatofocusing) buffers and the RP-HPLC buffers and fractionated by chromatofocusing and RP-HPLC using the PF-2D apparatus, leading to the separation of antigens in a 96-well plate. Since the antigens are enriched by immunoprecipitation, it is easier to visualize differences between the positive and negative cell lines in the fractionation profiles. The profiles can be visualized on a comprehensive map using specialized software such as the DeltaVue™ software. Specific fractions, distinctly different from the cell lines that do not express the antigen of interest can be chosen for further analysis.

### Tryptic Digests of 1D-Peak Fractions:

Fractions eluted from the chromatofocusing column typically contain large amounts of salts along with other chaotropic agents, present in the 1D elution buffer. Therefore, these fractions are preferably desalted by known methods and then subjected to tryptic digestion in-solution. The peptides obtained can be concentrated using, for example, μC18 column fractionation using Ziptips™. The concentrated peptides eluted from the μC18 column can be analyzed by LC-MS/MS for protein identification.

### Tryptic Digests of 2D-Fractions:

Fractions eluting from the 2D-column can be concentrated using a YM-10 microcon filter to remove excess acetonitrile in the fraction prior to proceeding with the in-solution tryptic digestion procedure to obtain peptides for LC-MS/MS analysis.

### Tryptic Digests of Immunoprecipitates:

Immunopurified proteins can be acetone precipitated to remove salts/elution buffers and reconstituted in buffer such as 10 mM Tris, 5 mM CaCl₂, followed by tryptic digestion. The peptides are reconstituted in CF-start buffer and fractionated to obtain 1D and 2D fractions. The peptides fractionated and differentially regulated are then reconstituted analyzed by LC-MS/MS to obtain protein ID.

Using the above approach of tryptic digestion at different stages of the purification/separation process, specific peptide antigens that are part of larger proteins (as for example surface antigens of membrane proteins) can be resolved allowing for a comprehensive peptide mapping.

Comprehensive mapping profile allows more precise comparisons of proteins that are differentially regulated in different cells and also allows for more accurate downstream processing.

Since peptide digestion has been performed before the final step of the multi-dimensional separation, time gap between 2-dimension fractionation (such as with the PF-2D) and MS analysis can be minimized. It will be appreciated that the peptide fragments thus generated can be analyzed for sites of posttranslational modification using mass spectrometry as described, for example in Protein digestion for mass spectrometry is generally described for example in Protein Sequencing and Identification Using Tandem Mass Spectrometry, M. Kinter and N. Sherman, John Wiley & Sons D. Desiderio and M. Nibbering eds., 2000 incorporated herein by reference.

### Mass Spectral Analysis and Protein ID

Direct analysis of protein complexes using LC-MS/MS after 1D and 2D-LC separations, can yield protein identification in a very short period of time. MS/MS fragmentation on the most intense ions in the MS spectra can yield amino acid sequence information that may be used to deduce the protein ID.

In the case where the digestion of proteins is in the initial stage of fractionation, groups of peptides from antigen-positive and antigen-negative cell lines, eluting at the same PI or hydrophobicity ratios (same fraction numbers), can be analyzed as sets and the results compared to identify the proteins (or peptides derived therefrom) differentially regulated in those particular groups.

### Materials and Methods

### Growth and Maintenance of Tumor Cell Lines

The cell lines in the study were purchased from ATCC and were cultured in accordance with the guidelines and recommendations of ATCC. Cells were harvested at 60-70% confluence with viability>90%. SKBR-3 and

HepG2 were the antigen-positive and antigen-negative cell lines, respectively, and anti-HER-2 was the test antibody.

### Microsomal Membrane Preparation (Plasma Membrane+ER Contaminants)

Cells were centrifuged at 2000 RPM for 3 min at 4° C.—wash with phosphage buffered saline (PBS) (twice), resuspend in 1 mL of 250 mM sucrose/1 mM EDTA/20 mM Hepes pH 8.0 (100×10⁶ in 1 mL)+protease inhibitors−0.6 ug/mL, homogenized with a Wheaton homogenizer (15×), centrifuged at 2000 RPM—3 min. in 2 mL Eppendorf tubes. The post-nuclear supernatant was transferred (PNS-1) to labeled tube on ice. The pellet was homogenized again with 1 mL of extraction buffer—centrifuge at 2000 RPM for 5 min. The post-nuclear supernatant (PNS-2) was collected and pool with the first supernatant and the pellet was discarded. The PNS was centrifuged at 5000 RPM (8000× g) for 10 min at 4° C. and the pellet (mitochondria) was discarded to retain the supernatant (post-mitochondrial supe) which was centrifuged at 43,000-55000 RPM (70,000-90,000× g) for 45 min at 4° C. The resulting supernatant is the cytosol which can be saved (5-10 μL) for protein assay—aliquot and stored at −80° C.

The collected pellet which corresponds to microsomal membrane fraction was resuspended in 20 mM Hepes pH 8.0+Octyl-β-glucoside(OBG)—500 uL+protease inhibitors—10 μL each of stock (Leupeptin, Pepstatin, Bestatin, PMSF and Aprotinin). After 2 min 200 uL of 10% SDS was added to the membrane solution and vortexed to stabilize the membranes. A 5-10 uL aliquot was saved for protein determination and remainder stored in aliquots at −80° C.

### Immunoprecipitation

To 0.5 mg of microsomal membrane preparation from tumor or normal cells 2 μL of stock protease inhibitors (Leupeptin, Pepstatin, Bestatin, PMSF and Aprotinin) was added followed by 900 μL of immunoprecipitation buffer in a 2 mL Eppendorf tube. Fifty (50) μg of anti-HER-2 antibody was added (exact volume added depends on the affinity of the antibody used) and the solution was placed on nutator overnight at 4° C. Eighty μL of 50% slurry i.e. anti-Human IgM/IgA-agarose./Protein G-agarose (Immunopure) was added and incubated for 2 h at room temperature on nutator. The sample was then centrifuged at 10,000 RPM on Biofuge for 1 min or at 5000 RPM for 3 min −4° C. The supernatant can be saved—unbound fraction—by adding 1 mL acetone and freezing at −20° C. (to precipitate proteins for gel analysis).

The pellet was resuspended (anti-Human IgM/IgA-agarose./Protein G-agarose) in 1 mL RIP-A buffer and incubated on nutator for 5 min at room temperature, centrifuged at 5000 RPM for 3 min and the supernatant was discarded. The sample was then eluted with 50 μL of 0.2M glycine pH 2.5 on nutator for 15 min at room temperature followed by centrifugation at 13,000 RPM for 15 min at 4° C. The supernatant was removed and added to the tube with 2 μL 1M TRIS pH 7.6 and was frozen at −20° C.

Immunoprecipitation Dilution Buffer: (1:9 Dilution)

| Dilution | Stock | | |
|---|---|---|---|
| 0.01% SDS | 0.25 | mL | 10% SDS |
| 1.1% TritonX-100 | 13.8 | mL | 20% Triton X-100 |
| 1.2 mM EDTA | 0.6 | mL | 0.5 M EDTA |
| 16.7 mM Tris pH 8.1 | 4.2 | mL | 1M Tris pH 8.1 |
| 167 mM NaCl | 8.35 | mL | 5M NaCl |
| Final Volume | 100 | mL. | |
| RIP-A Buffer; | | | |
| 25 mM HEPES | 5 | mL | 0.5M Hepes |
| 150 mM NaCl | 3.75 | mL | 4M NaCl |
| 0.5% Na deoxycholate | 0.5 | g | chemical stock |
| 1.5% SDS | 1.5 | mL | 10% SDS |
| 10% glycerol | 10 | mL | 100% stock |
| 1 mM EDTA | 200 | μL | 0.5M EDTA (RT) |
| 3 mM MgCl₂ | 300 | μL | 1M MgCl₂ |
| 1 mM DTT | 100 | μL | 1M DTT |

| | |
|---|---|
| 0.6 ug/mL - Leupeptin, Pepstatin, | 0.6 mg/mL (−20° C.) |
| Bestatin, Aprotinin. | |

### PF-2D™ Fractionation of SKBR-3 and HepG-2

The immunoprecipitated protein mixture/pre-fractionated antigen mixture was equilibrated with CF-start buffer in order to run on the PF-2D first dimension (chromatofocusing). Since the HER-2 antigen mixture contained mostly highly hydrophobic and proteins with Pi greater than 8.5, they were found to be insoluble and fractionated as the first peak (fraction number A2) in the chromatofocusing run. This fraction was clarified of all particulate material by high speed centrifugation. The clear supernatant was equilibrated with solvent A (0.1%TFA) in the ratio of 1:4, and fractionated on the HPRP column with a gradient of 0-100% acetonitrile containing traces of TFA.

### Fractionation Analysis Using ProteoVue™/DeltaVue™ Software

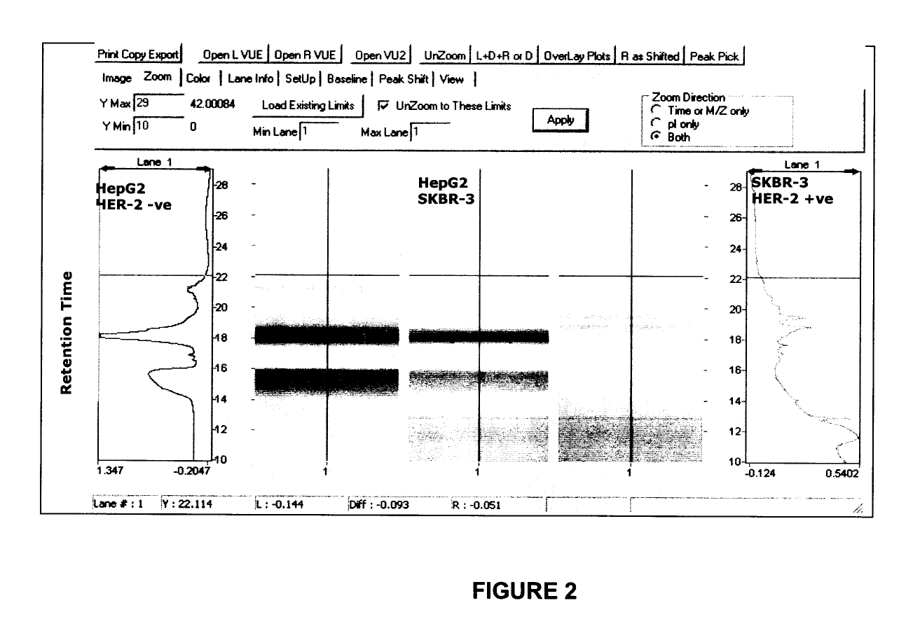

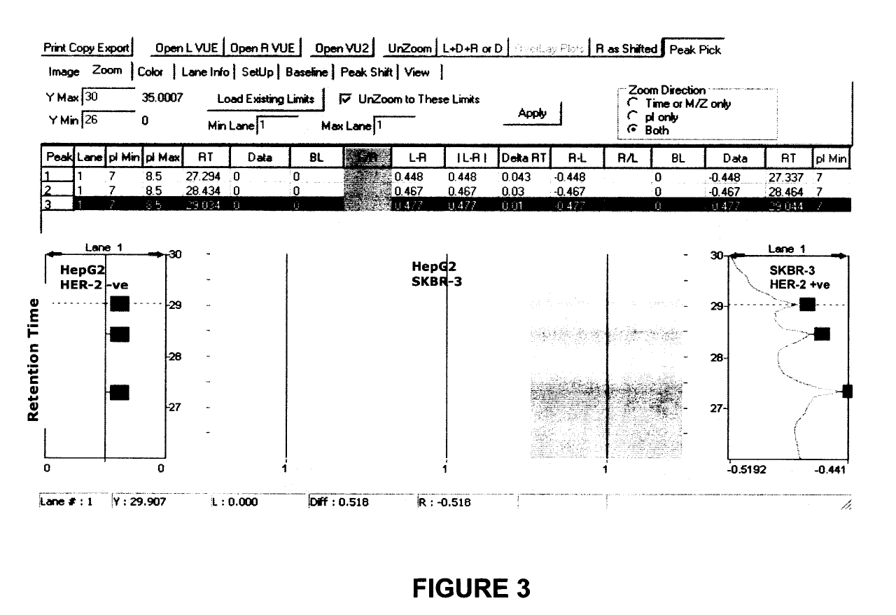

The chromatographic profiles obtained for the HPRP column fractions were imported into ProteoVue™ files to be formatted into an acceptable format for the final analysis on DeltaVue™. The analyses were combined for the antigen fractionation from both positive (SKBR-3) and negative (HepG-2) cell lines and formatted using ProteoVue® software to generate a comprehensive membrane protein map from each of the cell lines. A comparative profiling of differentially regulated proteins was thereafter generated on the DeltaVue™ software (FIGS. 2 and 3). The chromatographic profiles of the fractionation from both cell lines were converted from peaks to banding patterns making areas of differential expression more readily visible. Particular differentially expressed peaks/bands in the positive cell line could be focused for better resolution and analysis. FIG. 2 shows overall view of separation of HER-2 positive (SKBR-3) and HER-2 negative (Hep G2). The left and right spectra show differences in antigen profiles. FIG. 3 shows three distinct peaks eluting at about 27, 28 and 29 minutes, present only in HER-2-positive, SKBR-3 and absent in HER-2-negative Hep G2 cells.

### Dot Blot Assay

Fractions from the HPRP columns from both the cell lines were concentrated using microcon membranes and spotted on nitrocellulose membrane using the dot blot manifold. The fractionated proteins were probed with anti-HER-2 antibody and reaction measured with ECL.

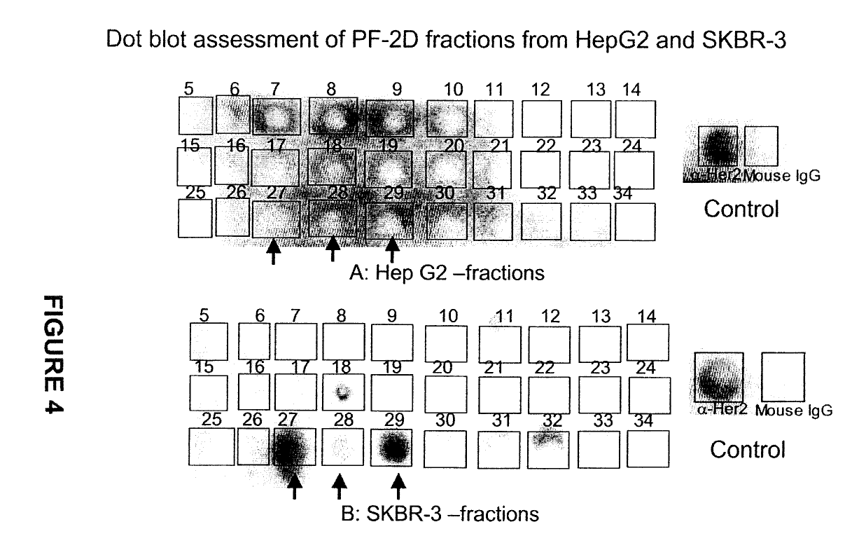

One blot for each cell line (34 fractions) was processed along with a positive control and a negative control (see FIG. 4). The positive and negative controls were SKBR-3 total membranes (5 μg) probed with anti-HER-2 (positive control) and mouse IgG (isotype-matched control), respectively. Positive fractions were subsequently analyzed by SDS-PAGE and Western blotting (FIG. 5). The protein fractions that were differentially regulated in HER-2 positive, SKBR-3, were concentrated and screened for the presence of HER-2 by SDS-PAGE and Western Blotting. Fraction 27, showed a positive signal at ˜180 kDa, which is the molecular weight of HER-2 antigen.

### Example 2

### Preliminary Characterization of VB1-050 Ag

The antigen recognized by the VB1-050 antibody showed a 58.62% (P-value 0.008) increase in binding upon deglycosylation. This increase in the binding of the antigen observed upon deglycosylation, suggests that the glycan moiety may partially mask the antigenic sites on the cell surface and that deglycosylation may be an essential step in the identification of the antigen.

### Immunoprecipitation

Equal amounts of membrane preparations from each of four positive cell lines (shown to bind the VB1-050 antibody), MCF-7, MDA-MB-435S, A-375, HepG2, and three negative cell lines (shown not to bind the VB1-050 antibody), Panc-1, Daudi and C-33A were deglycosylated with N-Glycanase and nutated with 40 μg VB1-050 and and isotype-matched control antibody (4B5-IgG) each in the presence of protease inhibitors with conditions mimicking in-vivo conditions. Immune complexes were centrifuged, washed with RIP-A lysis buffer and eluted with 0.2M glycine pH 2.5.

### Gel-Based Analysis and Western Blotting

Immunoprecipitates from all the above-mentioned cell lines were subjected to reducing and non-reducing conditions of sample preparation and were subsequently analyzed by SDS-PAGE and Western blotting. The resulting blots were probed with 4B5-IgG and VB1-050 simultaneously and corresponding secondary antibody conjugated to HRP, to visualize the immunoprecipitated proteins by chemiluminescence. A single band was detected at ˜50 kDa from VB1-050 immunoprecipitates on 1D-PAGE in all the cell lines and 2D-PAGE did not yield any result. No bands were detected with 4B5-IgG. Since this approach did not show any differentially expressed antigen, an alternative method for antigen identification was explored.

HTP-Antigen ID Using ProteomeLab™ PF-2D in Tandem with Nano-ESI-MS/MS

### PF2D Fractionation of HepG2, MCF-7, Panc-1 and C-33A

The pre-fractionated VB1-050 immunoprecipitates from membrane preparations were clarified of all particulate material by high speed centrifugation. The clear supernatant was equilibrated with Start buffer and fractionated on the chromatofocusing column in the first dimension. The peak fractions eluting at pH=7.4-7.6 was equilibrated with solvent A (0.1% TFA) in the ratio of 1:4, and fractionated on the HPRP column with a gradient of 0-100% acetonitrile containing traces of TFA.

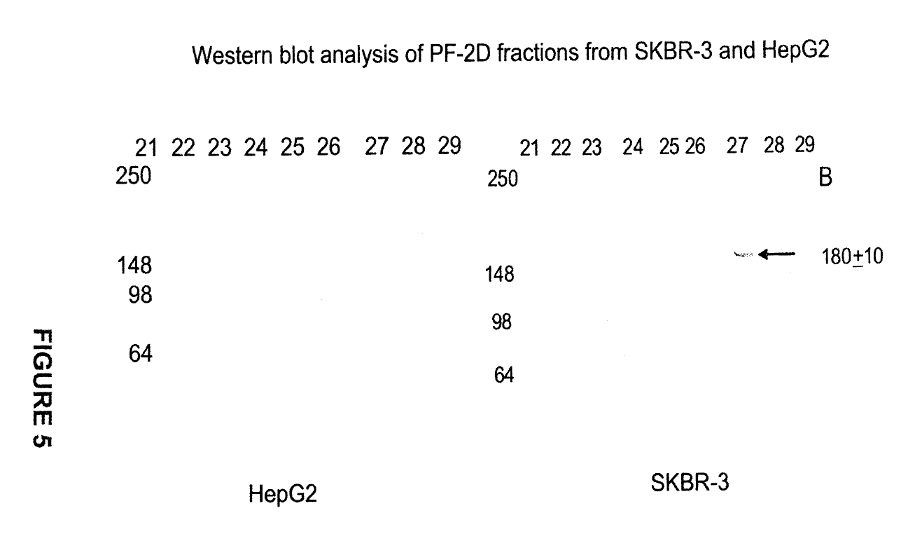

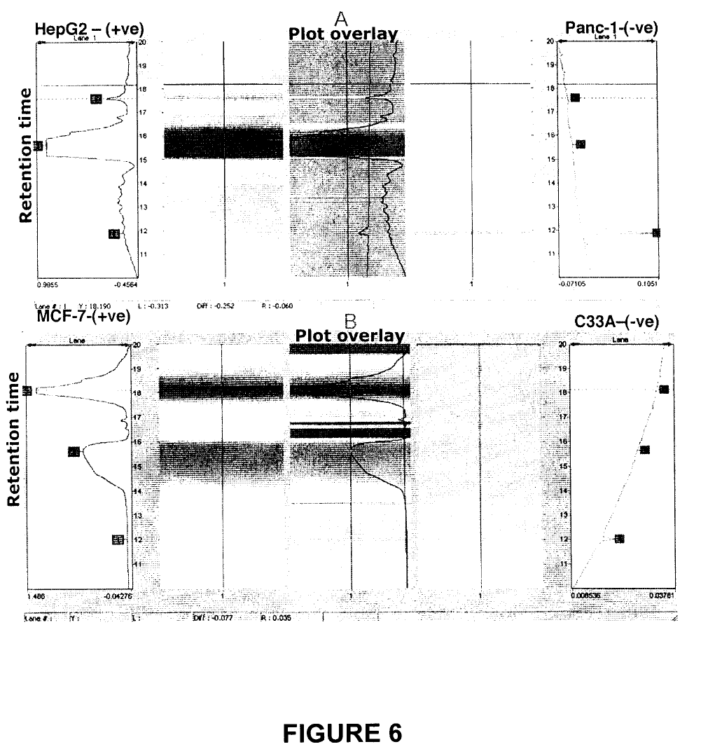

HepG2 and MCF-7 upon fractionation on the chromatofocusing column (CF), showed a single broad peak eluting at pH 7.4-7.6 as two fractions (constituting # B6 and B7) at 68 and 65 minutes, respectively. As observed in FIGS. 6A and B, HepG2 and MCF-7 membranes eluting off the HPRP column showed different separation profiles, entirely dependent on the presence of the VB1-050 reactive antigens. Two peaks were observed to be differentially regulated in the positive cell lines, that seemed to be negligible or totally absent in the negative cell lines, Panc-1 and C-33A membranes. On thorough analysis of the protein peaks present in the positive cell line (MCF-7 and HepG2), it was shown that the peaks elute from the RP-HPLC column with retention times of 15 and 18 minutes, respectively. These peaks were not observed in the antigen-negative cell lines (Panc-1 and C-33A). Instead, a single peak eluting earlier at 12 minutes was observed in the negative cell lines.

### Fractionation Analysis Using ProteoVue™/DeltaVue™ Software

The chromatographic profiles obtained for the HPRP column were imported into ProteoVue™ files to be formatted into an acceptable format for the final analysis on DeltaVue™. The analyses were combined for the antigen fractionation from both positive (HepG2 and MCF-7) and negative (Panc-1 and C-33A) cell lines and formatted using ProteoVue® software to generate a comprehensive membrane protein map from each of the cell lines. A comparative profiling of differentially regulated proteins was thereafter generated on the DeltaVue™ software. The chromatographic profiles of the fractionation from both cell lines were converted from peaks to banding patterns making areas of differential expression more readily visible. Particular differentially expressed peaks/bands in the positive cell line could be focused for better resolution and analysis. Overlaying the positive and negative plots obtained in each experiment showed that the over-expression of proteins was seen only in the positive cell lines (HepG2 and MCF-7) and these fractions were used for peptide extraction purposes.

### Peptide Extraction From Peak Fractions

Tryptic digestions were performed with sequencing grade trypsin in a 20-hour peptide extraction process finally resulting in the extraction of peptides that were analyzed on a QSTAR Pulsar-I (ESI-qTOF-MS/MS), equipped with a nanosource with a working flow rate of 20-50 nL/min. The peptides ionize and are detected as doubly, triply or quadruply charged molecules which are then refined to their respective masses. De-novo sequencing of the identified proteins was also performed whenever possible. Peptides were extracted from both positive and negative cell lines to ensure it was the right antigen. Peptide masses extracted from the mass spectra were used directly to identify the antigen according to the MOWSE scores obtained on protein databases that are accessible through the MASCOT search engine.

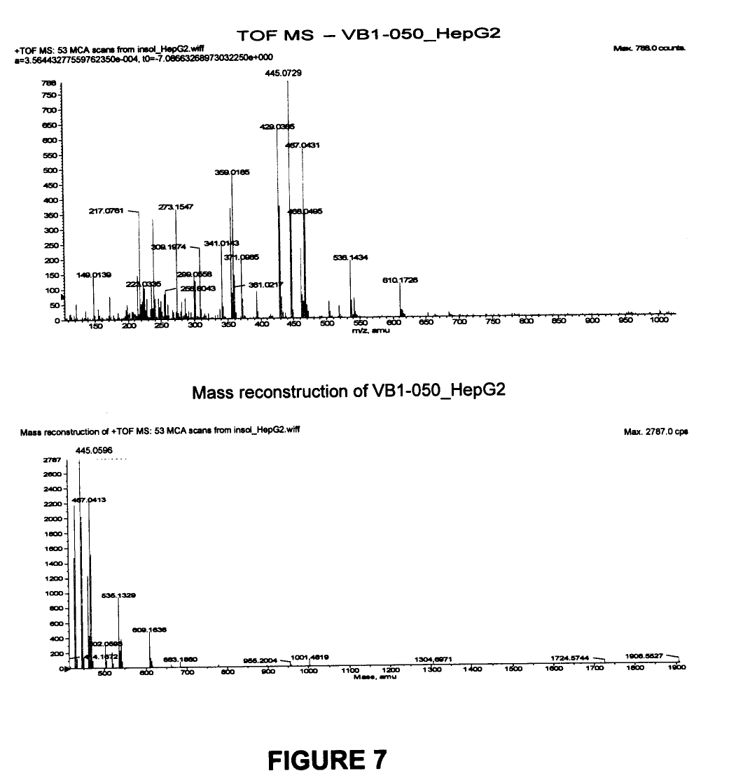

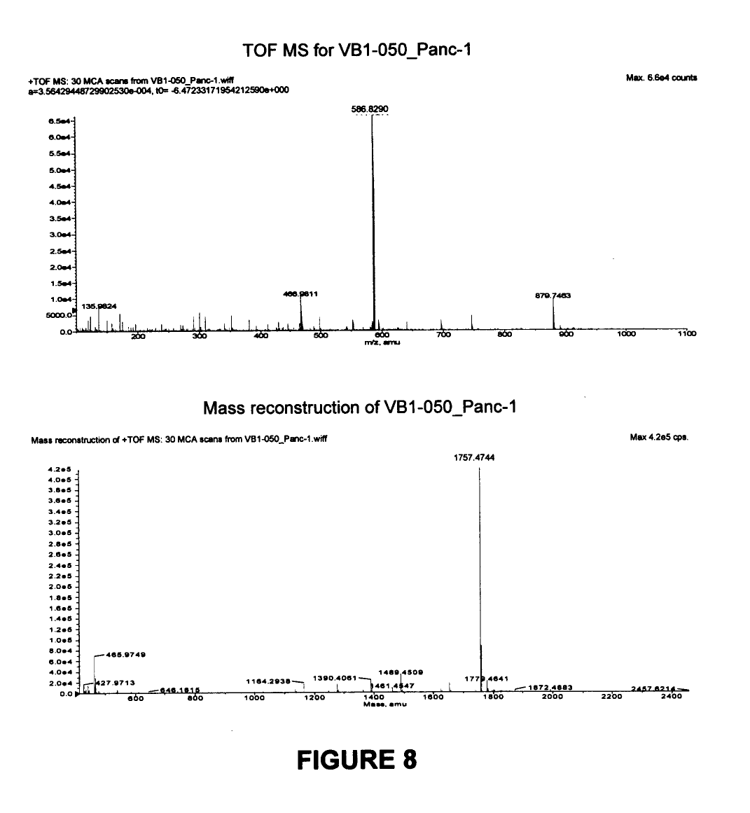

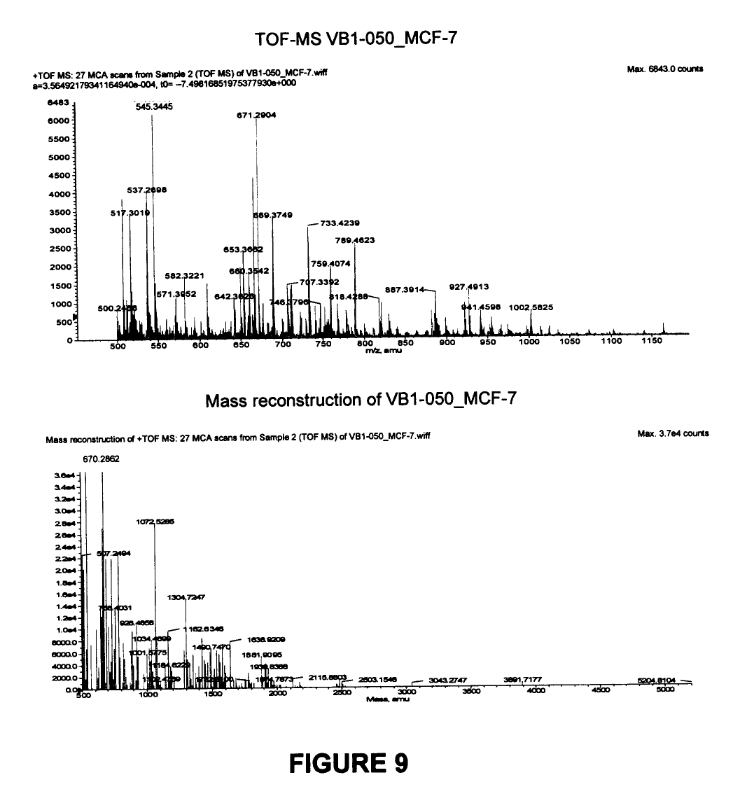

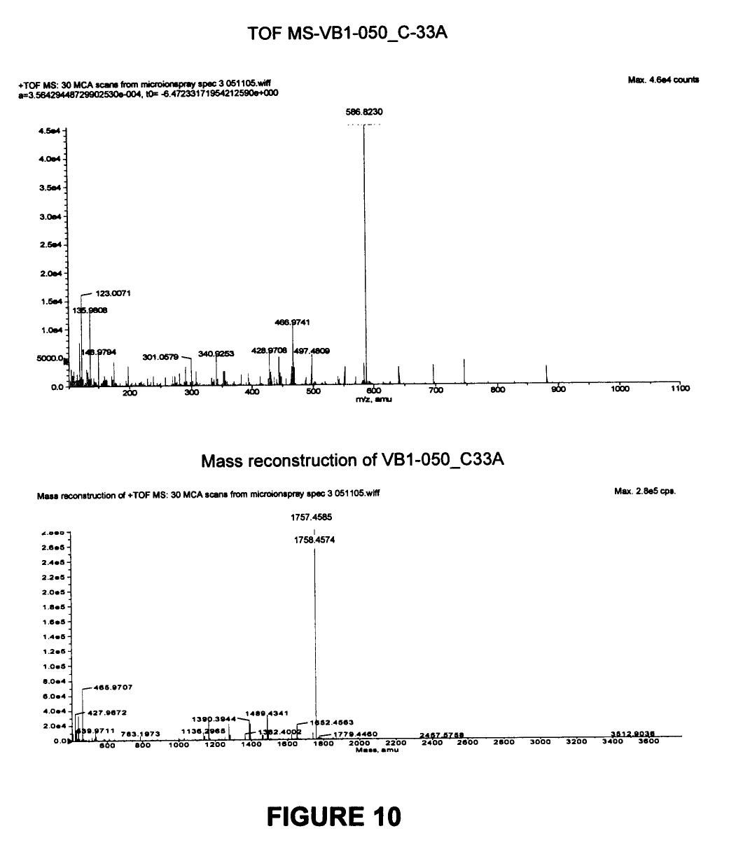

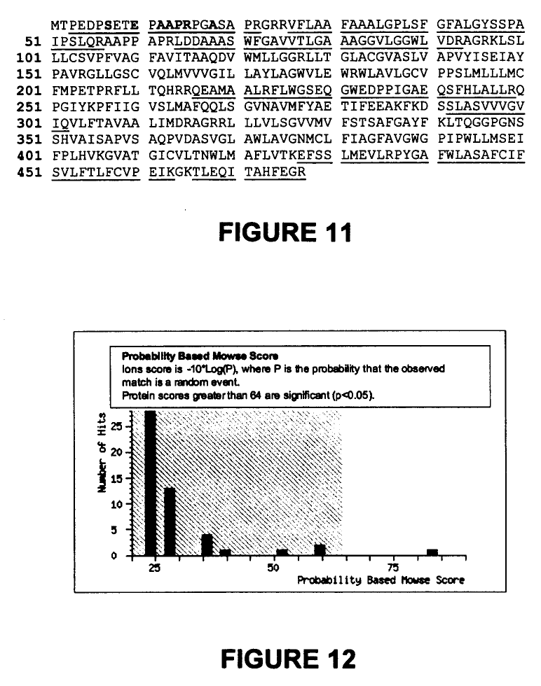

Peptides were extracted post-tryptic digestion from the peak, fractions eluting at 15-18 minutes, from all four samples (MCF-7, HepG2, Panc-1 and C-33A) and subjected them to MS analysis. In addition to fractions eluting at 15, 18 minutes, fractions eluting at the 12^{th} minute from positive and negative cell lines were also processed simultaneously. FIGS. 7-10 show results of the TOF-MS scans of the peptides obtained from the cell lines. As seen in FIG. 11, one single protein was identified from both the positive cell lines corresponding to glucose transporter-8 but was undetectable in the negative cell lines. The difference in elution between the two peaks (15 vs 18 minutes) could be attributed to changes in glycosylation or other post-translational modifications.

### Mass Spectral Analysis

Peptide analysis was done in two ways:

All the peptides recovered and reconstructed to their right masses were used directly in a peptide mass fingerprinting step to obtain an ID for the protein.

Peptides that were abundant and well ionized were chosen for further MS/MS ion fragmentation, wherein, the ‘y’ and ‘b’ ions were used to deduce their primary structure. These sequences were then searched for homologies in the protein database for protein ID.

Peptides ionize and are detected as doubly, triply or quadruply charged molecules, on a LC-MS/MS system as opposed to detection as singly charged on Matrix assisted ionization such as in MALDI. Differentially charged peptides were thereafter refined to their respective masses, in the mass reconstruction step. These peptide masses were then directly analyzed by a matrix science based mascot search engine for antigen ID. Peptide masses extracted from the mass spectra were used directly to identify the antigen according to the MOWSE scores obtained on protein databases that are accessible through search engines such as MASCOT, SEQUEST, and Prospector. Since the QSTAR-pulsar-I purchase includes the purchase of license from Pepsea server for most recent protein database additions, and is compatible with MASCOT, this search engine was selected for all protein searches.

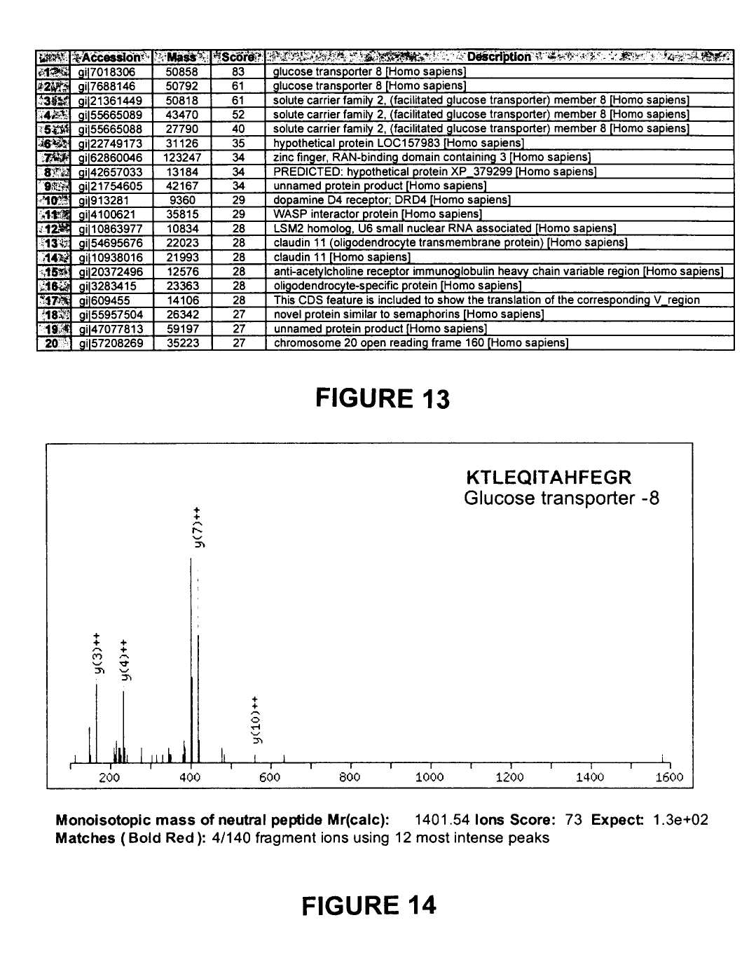

The list of peptides recovered and their mapped positions to the sequence from Glucose Transporter 8 are as given in FIGS. 11, 12 and Table 1. All peptides represented were obtained by de novo sequencing. FIG. 13 identifies Glucose Transporter 8 as the antigen.

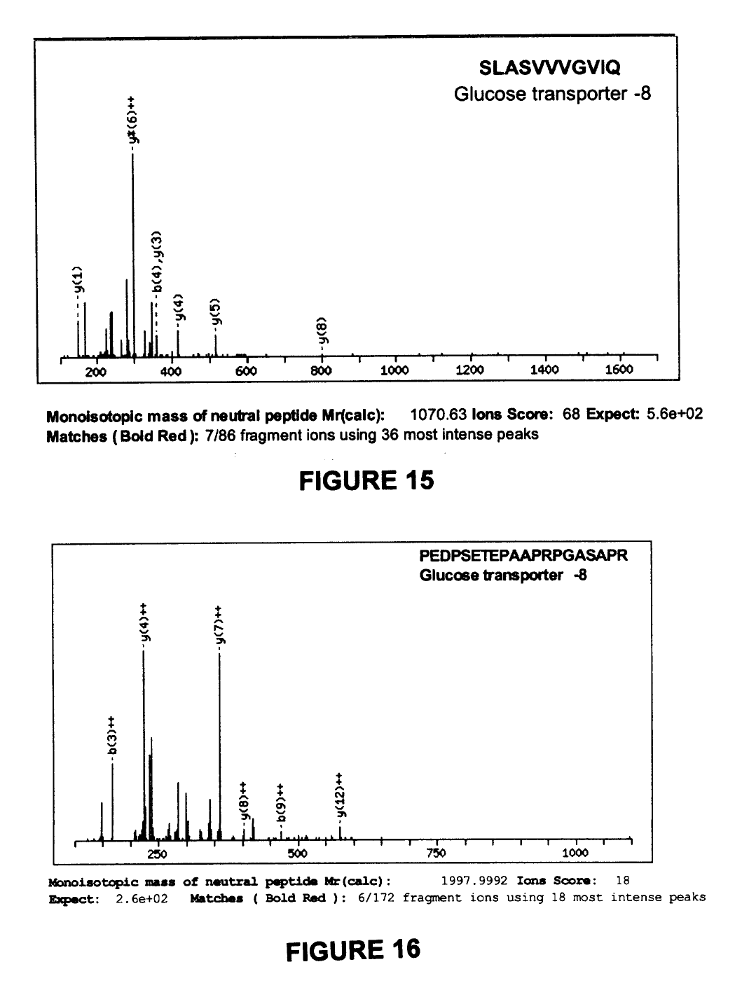

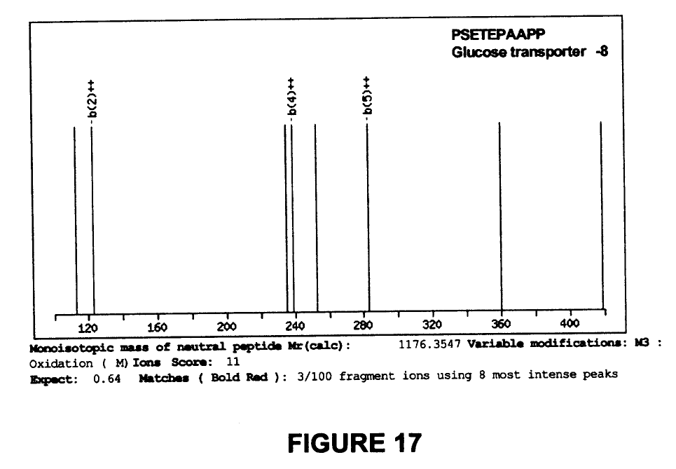

MS/MS fragmentation of four of the peptides (1401.54-466.600000, 3+; 1070.785448-536.400000, 2+; 1998.272862-667.098230, 3+; 1176.185448-589.100000, 2+) gave rise to the fragment ions shown in FIGS. 14-17 that mapped to peptides from Glucose Transporter 8. Since these 2 peptides were all detected in TOF-MS, these peptides were used for MS/MS ion fragmentation apart from the peptides derived from mass fingerprinting. A discrete nanospray head installed on a nanosource was used for the purpose. The collision energy was 48V, curtain gas and CAD gas were maintained at 25 and 6, respectively, and the sample allowed to cycle for 1.667 minutes (100 cycles) to obtain stable mass ion fragmentation. Peptides derived from the spectra clearly matched the sequence on Glucose Transporter 8, therefore were pulled down as major hits. The ion fragmentation data further confirm the identity of Glucose Transporter 8 as the cognate antigen for VB1-050.

Peptide mass fingerprinting and MS/MS fragmentation of the antigen-positive fractions revealed the identity of Glucose transporter-8/GLUTX1/SLC 2A8 gene product as the cognate binding antigen for VB1-050. Glucose transporter-8 is a ˜50 kDa type-II transmembrane protein, with N-terminus inside the cell. 34% sequence coverage was obtained from the peptides that were recovered in-house. Cell lines selected positive by flow show the presence of the antigen upon immunoprecipitation. MS/MS analysis of two peptides, 1070.785, appearing as a doubly charged molecule (536.40000, 2+); 1401.54, appearing as a triply charged molecule (466.60000, 3+), identified two peptide sequences, SLASVVVGVIQ (292-303) and KTLEQITAHFEGR (466-477), respectively, clearly matched the protein sequence corresponding to Glucose transporter-8.

MS/MS sequencing of two additional peptides recovered from MCF-7, 1176.3547 and 1997.9992, mapped sequences with 68.2% homology to corresponding peptides from GLUT8 with changes in amino acids at seven positions, i.e., 7, 10, 12-15, 18. Glut-8 has generally been recognized as an intracellular protein with the potential for membrane localization. However, the regulation of the translocation from cytosol to membrane has yet to be established. Shin et al. (2004, J. Neuro. Res. 75: 835) introduced a LL to AA mutation at positions 12, 13 and reported that this mutation resulted in the constitutive localization of GLUT8 from cytosol to the plasma membrane.

### Example 3

### Experimental Design

Melanoma cell line (A-375), glioma cell lines (U118MG and U87MG), breast cancer cell line (MDA-MB 435S), pancreatic cell line, (PANC-1) and T-cell line (Daudi) were used in the study (Table 2). These cell lines were selected based on the results of tumor cell line profiling by flow cytometry.

### Growth and Maintenance of Tumor Cell Lines

The cell lines in the study were purchased from ATCC and cultured in accordance with the guidelines and recommendations of ATCC. Cells were harvested at 90% confluence with viability>90%.

### Preliminary Characterization of the Antigen Binding to VB3-011

Preliminary characterization data was obtained from experiments designed to assess the feasibility of the gel-based approach by dot blot assays; and from experiments performed to determine the nature of the epitope associated with the antigens.

The data from these experiments classified the VB3-011 antigen as a “non-blottable” antigen with a glycan epitope, i.e., the epitope involved in binding to VB3-011 on the antigen was definitely glycosylated. It should be noted that the antigen could be glycosylated at sites other than the binding site as well.

### VB3-011 Ag Enrichment and Purification

The preliminary data from the blottability study specified a lectin-based purification method as the best antigen preparation method for the antigen recognized by the VB3-011 antibody. Extensive experimentation on the cell surface epitope determination revealed that VB3-011 reacted with at least three soluble forms of CS (chondroitin sulphate); two of these (CSB and CSE) have limited tissue distribution. As such, most of the antibody reactivity could be attributable to CSA and to a lesser extent hyaluronic acid, thereby identifying CSA or related glycans as key reactive moieties of the antigen molecule.

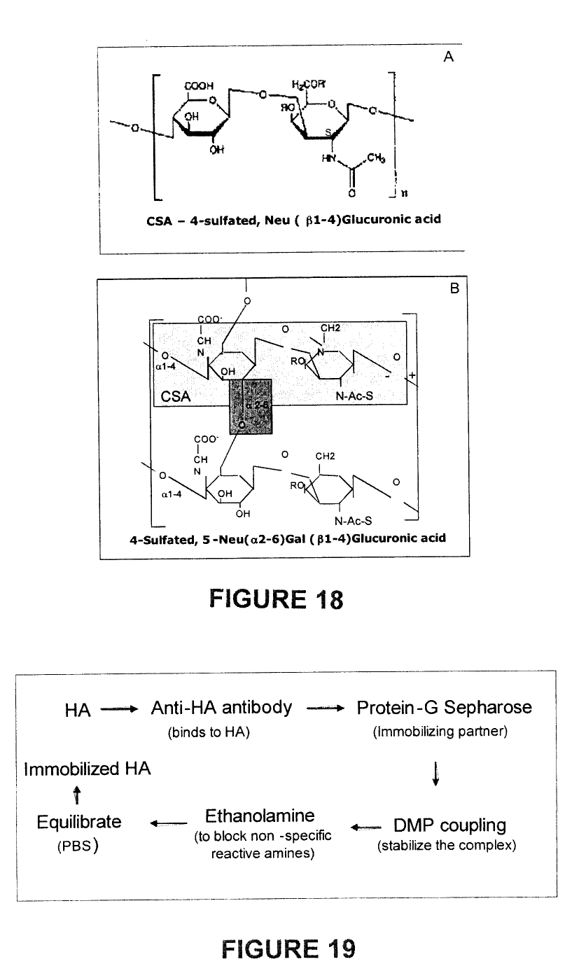

Chondroitin sulphate A (CSA) is made up of linear repeating units containing D-galactosamine and D-glucuronic acid. The amino group of galactosamines in the basic unit of chondroitin sulfate A is acetylated, yielding N-acetyl-galactosamine; there is a sulfate group esterified to the 4-position in N-acetyl-galactosamine (FIG. 18A) (Suguhara K et al. Structural studies on sulfated glycoproteins from the carbohydrate protein linkage region of Chondroitin 4 sulfate proteoglycans of swarm rat chondrosarcoma.

Demonstration of the structure Gal (4-O-sulfate) beta 1-3 Gal beta 1-4 XYL beta 1-O-Ser, 1988. J. Biol. Chem. Vol. 263:10168-10174; Suguhara K et al. Structural studies on sulfated oligosaccharides derived from the carbohydrate protein linkage region of chondroitin sulfate proteoglycans of wheat cartilage, 1991. Eur. J. Biochem. Vol. 202:805-811; Prydz K and Dalen K T. Synthesis and sorting of proteoglycans, 2000. J. Cell Sci. Vol. 113:193-205). When these linear repeating units get cross-linked (α 2-6) at branch points at C2 of the second and C6 of the first carbon chains, such that a single unit of glycan representing more than one linear chains of CSA are present, except for the sulfation, it resembles the glycan, Neu5Ac (α 2→6) Gal(β 1→4) Glucuronate, recognized by HA (FIG. 18B).

Two or more CSA molecules when cross-linked together resemble the glycan—Neu5Ac (α 2→6) Gal (β 1→4) Glucuronate, recognized by Hemagglutinin (HA), still retaining the identity of the epitope, CSA. Azumi et al., (1991) showed that the activity of a hemagglutinin isolated from hemocytes of the ascidian, Halocynthia roretzi was inhibited by heparin, chondroitin sulfate, and lipopolysaccharide (LPS), but not by mono- and disaccharides such as N-acetyl-galactosamine, galactose, and melibiose. The hemagglutinin showed binding ability to heparin, chondroitin sulfate and LPS, as demonstrated by heparin-Sepharose chromatography and centrifugation experiments, respectively (Ajit Varki et al. 1998. Essentials of Glycobiology). Similarly, a Hemagglutinin from mycobacterium was shown to bind to heparan sulfate and Hemagglutinin from Hemophilius influenzae binds to CSA with an additional α 2-6 linkage (Azumi K et al. A novel LPS binding hemagglutinin isolated from hemocytes of the solitary ascidian, Halocynthia roretzi: it can agglutinate bacteria, 1991. Dev. Comp. Immunol. Vol. 15(1-2):9-16; Menozzi F D et al. Identification of a heparan-binding hemagglutinin present in Mycobacteria, 1996. J. Exp. Med., Vol. 184(3):993-1001). Heparan sulfate and Chondroitin sulfate A differ in C5 epimerization. Therefore, a new reagent that would enable lectin-based purification was generated as follows. Recombinant HA was immobilized to anti-HA antibody by coupling with Dimethylpimelimidate (DMP), such that when used as an IP agent, HA recognizes the antigenic epitope on the cell surface. Membrane preparations were affinity purified with immobilized-HA, and the eluates subjected to SDS-PAGE and WB analysis, subsequently probed with VB3-011 antibody.

### Lectin-Based Purification

Recombinant HA molecule that binds specifically to the glycan—Neu5Ac (α 2→6) Gal (β 1→4) Glc, was made to bind to anti-HA antibody for 2 hours at room temperature on the nutator, followed by binding of the HA-anti-HA complex to Protein-G-sepharose. This was followed by a centrifugation step to get rid of the unbound fraction. The immobilized complex was then cross-linked using Dimethylpimelimidate (DMP) that is known cross-link proteins present in close proximities. The excess or unused cross-linker and the unbound material were removed by a brief centrifugation step. The non-specific amine groups that could have arisen as a by-product of the cross-linking step were neutralized with Triethanolamine for two hours at room temperature. The lectin-based reagent thus created was washed thoroughly with PBS and stored with PBS containing 0.05% NaN3 at 2-8° C. Apart from the HA-reagent, Con-A-agarose and WGA-agarose were also used as affinity purification reagents to detect better antigen recovery.

A minimum of 500 μg membrane protein was used for the lectin-based purification. A pre-clearing step using protein-G sepharose alone was the first step in the purification of the antigen prior to the addition of the reagent. A total of 15-20 μL of the reagent was used as the precipitating agent in the mixture. The antigen-lectin mixtures were nutated overnight at 4° C. using buffer conditions that mimicked physiologic conditions (i.e. pH and salt concentration were adjusted to be similar to physiological conditions). Care was taken to ensure that protease inhibitors were used in every step of the antigen isolation process.

Antigen-lectin complexes were centrifuged, washed with RIP-A lysis buffer and eluted with 0.2 M glycine pH 2.5. Supernatants representing the unbound fractions were stored to test the proteins that were not isolated by affinity purification. The procedure was carried out on two glioma cell lines (U118MG and U87MG), one melanoma cell line (A-375), one epithelial cell line (MDA-MB-435S) and two negative cell lines (Panc-1; and Daudi).

### Gel-Based Analysis and Western Blotting

1D-PAGE: The purified proteins were subjected to reducing conditions of sample preparation and were subsequently analyzed by SDS-PAGE/Western Blotting. When reducing conditions were used, the isolated antigens were treated with sample buffer containing 1% β-mercapto ethanol at 65° C. for 15 minutes. The resulting blots were probed with VB3-011 and corresponding secondary antibodies conjugated to HRP, to visualize the immuno-purified proteins by chemiluminescence.

2D-PAGE: The purified proteins were separated by two-dimensional gel electrophoresis to resolve any protein stacking effect that may have occurred in the 1D-PAGE analysis. The 2D-gel electrophoresis resolved proteins according to their isoelectric points (pl) in the first dimension and on the basis of their molecular weights in the second dimension. Proteins thus resolved were transferred to nitrocellulose membranes, overnight, and processed as in the case of 1D-PAGE. Western blots were probed with VB3-011 and reacting proteins visualized by chemiluminescence.

### Peptide Extraction and Antigen ID

Peptide extraction from in-gel and in-solution fractions: Tryptic digestions were performed with sequencing grade trypsin in a 20-hour peptide extraction process finally resulting in the extraction of peptides that were analyzed on a QSTAR Pulsar-I (ESI-qTOF-MS/MS), equipped with a nanosource with a working flow rate of 20-50 nL/min. The peptides ionize and are detected as doubly, triply or quadruply charged molecules which are then refined to their respective masses. De-novo sequencing of the identified proteins was also performed whenever possible. Peptides were extracted from both positive and negative cell lines to ensure it was the right antigen. Peptide masses extracted from the mass spectra were used directly to identify the antigen according to the MOWSE scores obtained on protein databases that are accessible through the MASCOT search engine. Peptides were extracted both from gel slices and in-solution (U118MG, U87MG, A-375, 435S) and subjected them to MS analysis.

### Results

### HA Reagent Immobilization

Recombinant HA molecule is not an antibody and therefore does not bind to protein-G-sepharose directly as an immobilizing partner. In order to make it possible for this antibody to be functional in antigen purification processes, HA was bound to anti-HA antibody that would bind specifically to HA, the molecule was immobilized using protein-G-sepharose in a sequential manner. This would not only immobilize the complex but would block any non-specific interaction that could arise from the presence of the anti-HA, as shown schematically in FIG. 19. The immobilized HA-anti-HA complex was thereafter stabilized using Dimethyl pimelimidate, a cross-linking agent that maintained the proximities of the various reactants. The final complex generated a few reactive amines in the process, other than the reactive binding site on the HA molecule. These reactive groups were blocked permanently using 1M triethanolamine, thus ensuring the maximal exposure of the reactive site on the HA molecule.

### Lectin-Purification

All precipitation reactions were performed with pre-cleared proteins. Longer incubation times were used to minimize non-specificity and enhance the stability of lectin-antigen complexes. Six cell lines (A-375, U118MG, U87MG, MDA-MB-435S, Panc-1 and Daudi) were used in the study. Reducing conditions for sample preparations were employed prior to the resolution of the antigens isolated on SDS-PAGE. The Western blots were probed with VB3-011 to ensure that the antigen purified is the cognate binding partner for VB3-011.

### 1D-PAGE/Western Analysis

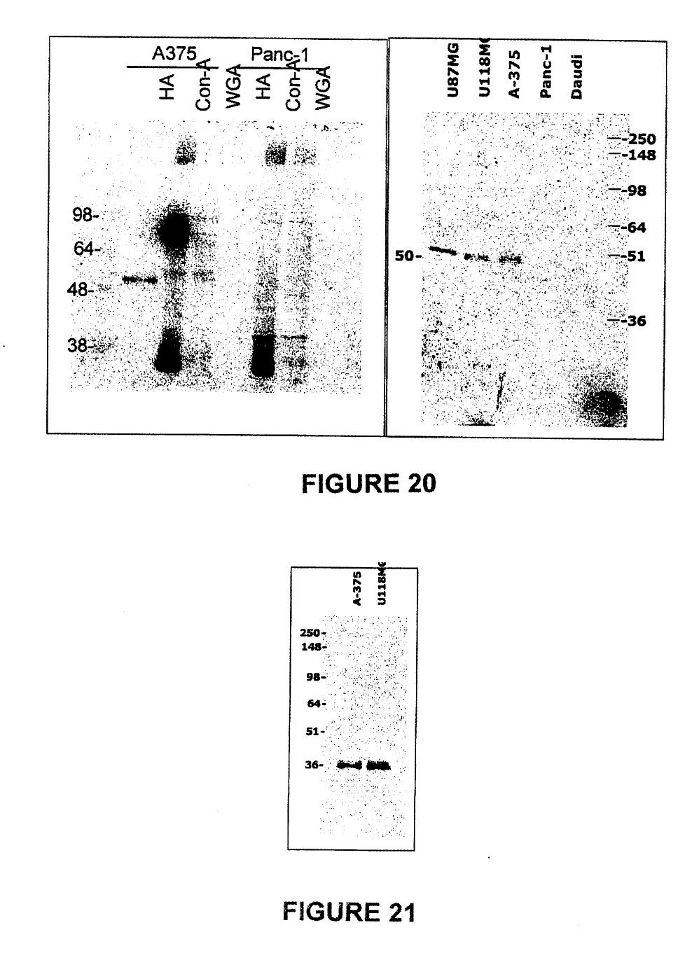

When HA reagent was used, only one specific band was detected after separation on a 1D-PAGE at ˜50 kDa under reducing conditions (FIG. 20A) in antigen-positive cell line (A-375), that was absent in the negative cell line (Panc-1). Non-specific interactions were observed with Con-A and WGA lectins indicating that the glycan present on the VB3-011 antigen was the one recognized by HA. Glioma cell line (U118MG and U87MG) also showed the presence of a single band at ˜50 kDa when purified using the HA reagent (FIG. 20B). When samples were allowed to sit at room temperature for 1 hour prior to their separation on SDS-PAGE, a predominant band at ˜36 kDa and a faint 50 kDa band were observed in antigen-positive cell line (A-375, U118MG and U87MG) indicating that the peptide component of the antigen could be ˜36 kDa (FIG. 21).

### 2D-PAGE Analysis

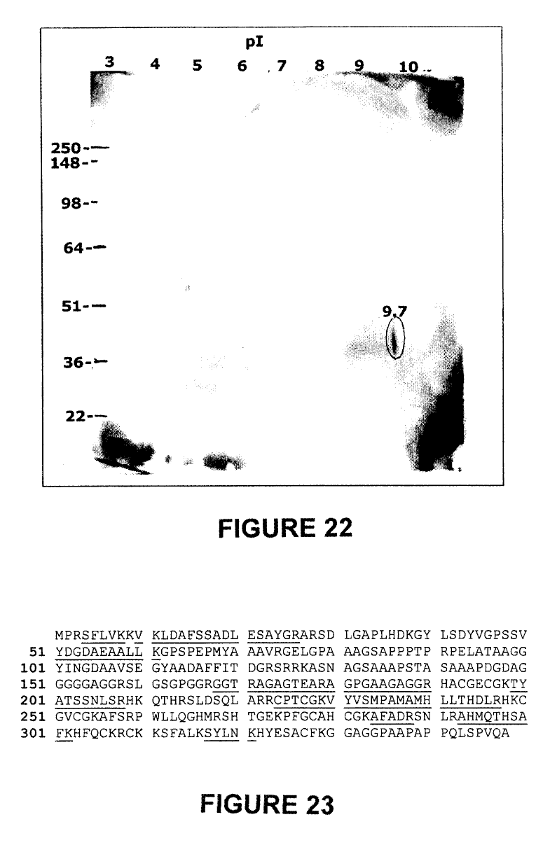

In order to determine isoelectric points (pl) and assess the possibility of protein stacking in the 1D-PAGE analysis, the antigens purified by HA were separated on two-dimensional polyacrylamide gel electrophoresis (2D-PAGE), where the separation in the first dimension is on the basis of pl and the second dimension on the basis of molecular weight. The gels were then transferred to nitrocellulose membranes and subjected to standard Western blotting processing. Since the amounts required for the detection of proteins on a 2D gel is ˜4 times higher than the requirement for a 1D gel, purified antigens from 4 separate immunoprecipitation reactions were pooled together for one 2D-PAGE analysis. Two separate gels were processed simultaneously for Western blot analysis to ensure that the proteins detected on the Coomassie stained gels are the same as those observed in the Western blots. The 2D Western blots were probed with VB3-011 and detected by ECL (chemiluniscence). As can be seen in FIG. 22, one single spot was detected at ˜36 kDa/pl=9.7±0.2.

### Peptide Extraction and Protein Analysis

A-375, U 87MG and U118 MG membranes were used to immunopurify antigen(s) that bind specifically to VB3-011. A ˜50 kDa band was observed in all three cell lines as shown in FIG. 20. The protein bands were excised from the coomassie stained gels and used in-gel digestion to extract peptides for MS analysis.

Proteins from 1D-gel band and 2D-spots were digested with trypsin to release them from the gel and analyzed on a reverse-phase LC-MS/MS system. The identities of the proteins were revealed by database analysis using bioinformatic tools. Raw data included peptides obtained as listed in the TOF-MS spectra, MS/MS fragmentation data, and a list of suggested proteins including contaminants that do not match the pl or the molecular weight of the protein isolated. To obtain the analysis MS/MS spectra were submitted directly to Mascot search engines available at www.Matrixscience.com.

### Mass Spectral Analysis

Peptide analysis was done in two ways:

All the peptides recovered and reconstructed to their right masses were used directly in a peptide mass fingerprinting step to obtain an ID for the protein.

Peptides that were abundant and well ionized were chosen for further MS/MS ion fragmentation, wherein, the ‘y’ and ‘b’ ions were used to deduce their primary structure. These sequences were then searched for homologies in the protein database for protein ID.

Peptides ionize and are detected as doubly, triply or quadruply charged molecules, on a LC-MS/MS system as opposed to detection as singly charged on Matrix assisted ionization such as in MALDI. Differentially charged peptides were thereafter refined to their respective masses, in the mass reconstruction step. These peptide masses were then directly analyzed by a matrix science based mascot search engine for antigen ID. Peptide masses extracted from the mass spectra were used directly to identify the antigen according to the MOWSE scores obtained on protein databases that are accessible through search engines such as MASCOT, SEQUEST, and Prospector. QSTAR-pulsar-I was used and selected for all proteion identities, because it includes the most recent protein database additions from Pepsea is compatible with MASCOT.

### Analysis of 2D Spot

Protein spot excised from the 2D-gel were found highly homologous to Scratch. The pl and the molecular weight clearly matched the Mammalian Scratch. A total of 37% sequence coverage with 15 matching peptides, each peptide showing 100% homology to the original protein was recovered (See FIG. 23).

### Analysis of the 50 kDa Band Purified From the Glioma and Melanoma Cell Lines

The data obtained from the mass spectra of all three cell lines, (U87MG, U118MG and A375) point towards Mammalian scratch as the antigen that binds to VB3-011. Of all the cell lines screened, glioma cell lines (U87MG and U118MG) showed the highest scoring identities. A-375, a melanoma cell line also showed an over-expression of the antigen. Apart from the above mentioned cell lines, epithelial cell lines such as MDA-MB-435S, PC-3, A-549 and CFPAC-1 were also screened in the same manner, but except for MDA-MB-435S, which showed the presence of a truncated version of Scratch, i.e., 17.823 kDa protein qi|15928387, with 100% homology to sequences 158-366 of the original scratch molecule. The membrane preparations from each of these cell lines were used to affinity purify the VB3-011 antigen using the HA-reagent. The other epithelial cell lines tested showed no detectable proteins.

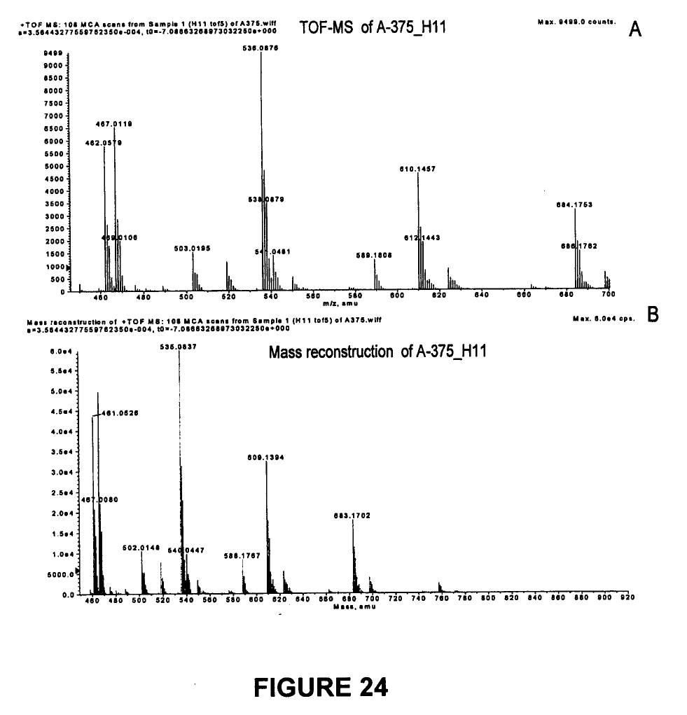

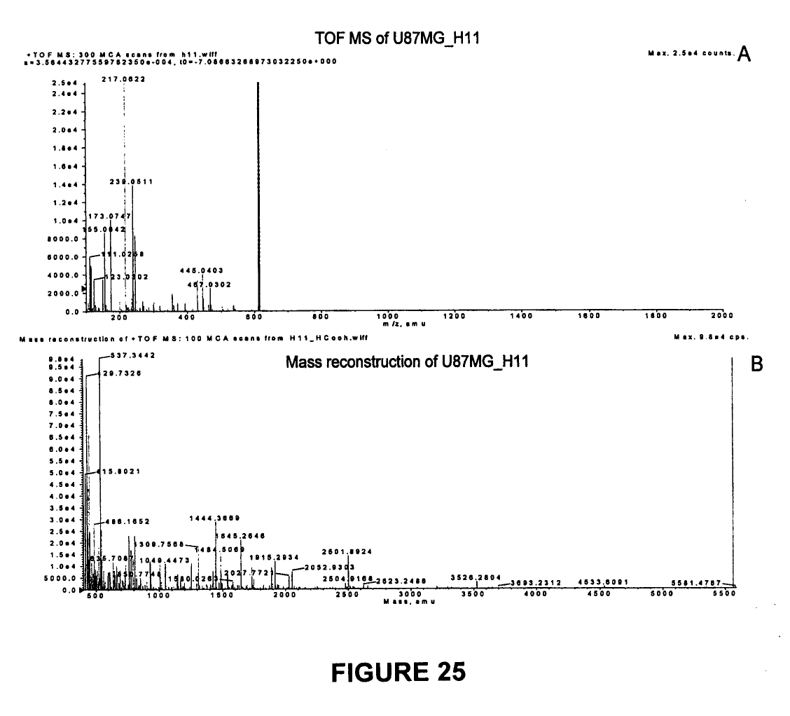

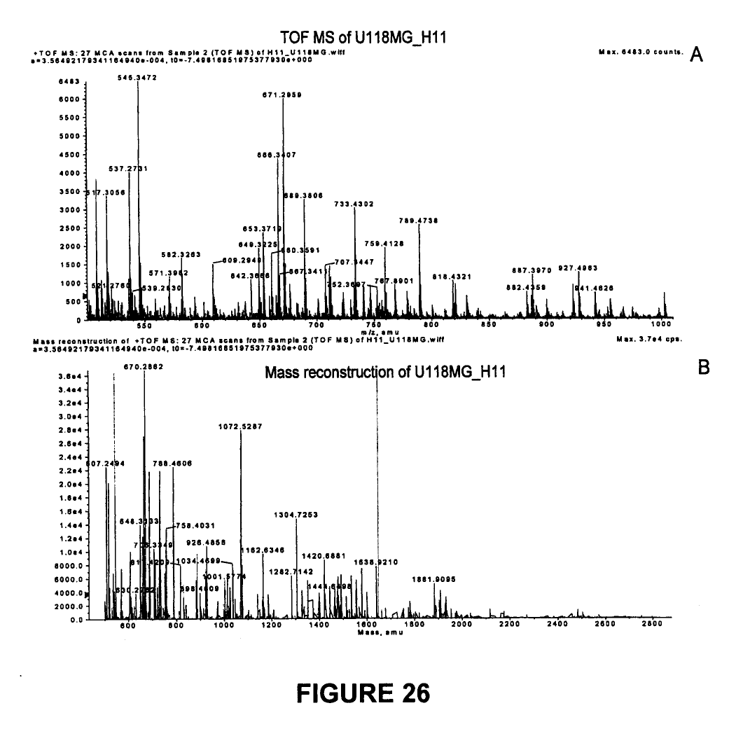

TOF-MS scans were obtained both on a manual mode and an IDA mode to recover the maximum number of peptides for a significant ID. See FIGS. 24-26.

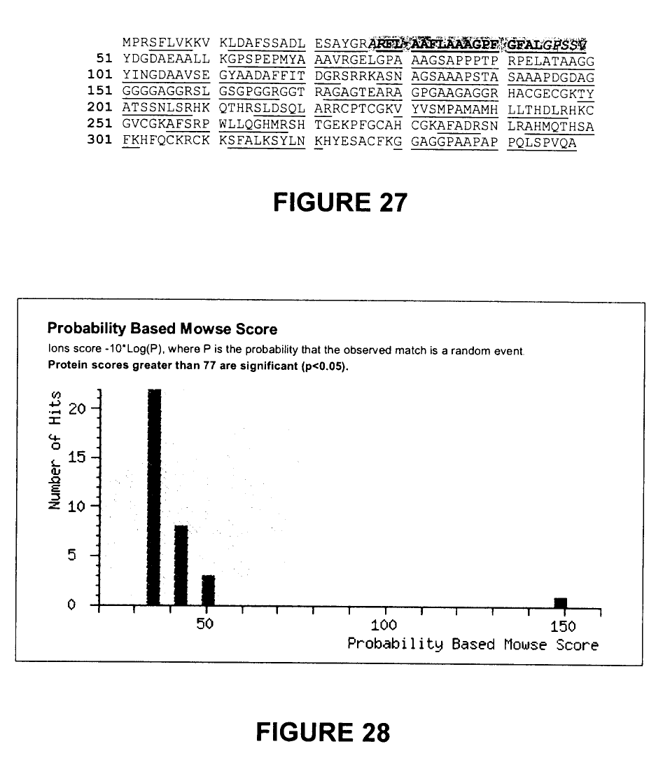

The list of peptides recovered and their mapped positions to the sequence from Mammalian Scratch are as given in FIG. 27 and Table 3. All peptides represented were obtained by de novo sequencing.

### MS/MS Fragmentation of Peptide 2402.1206 and 2134.9614

A discrete nanospray head installed on a nanosource was used for the purpose. The collision energy was 48V, curtain gas and CAD gas were maintained at 25 and 6, respectively, and the sample allowed to cycle for 1.667 minutes (100 cycles) to obtain stable mass ion fragmentation. MS/MS fragmentation of two of the peptides (2402.978172-802.00000, 3+; 2134.985448-1068.500000, 2+) gave rise to the fragment ions shown in FIGS. 30 and 32. While one of the peptides, ‘PELATAAGGYINGDAAVSEGYAADAF’ from peptide mass 2402.97812, mapped 100% to a sequence from Scratch, peptide, RFLAAFLAAAGPFGFALGPSSV, from peptide mass 2134.985448, showed 100% homology in the flanking sequences but not with the sequence in the middle, indicating an identification of a novel sequence. The presence of this sequence is responsible for the only transmembrane domain available on the protein. Mammalian scratch sequence available in the database is a result of conceptual translation and does not have any transmembrane domains in the sequence. The protein sequence recovered shows 67% homology to the Scratch protein available in the database and indicative of being present on the cell surface due to the presence of a transmembrane domain. Rest of the peptides derived from the spectra clearly matched the sequences from Mammalian Scratch, and therefore were pulled down as major hits. The ion fragmentation data further confirm the identity of a novel form of Scratch as the cognate antigen for VB3-011.

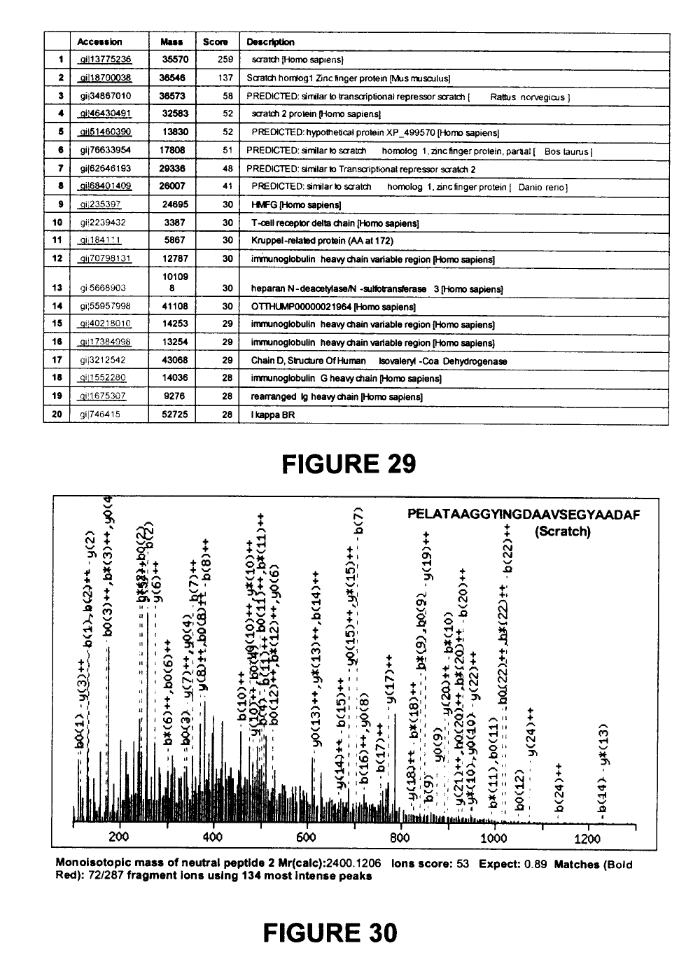

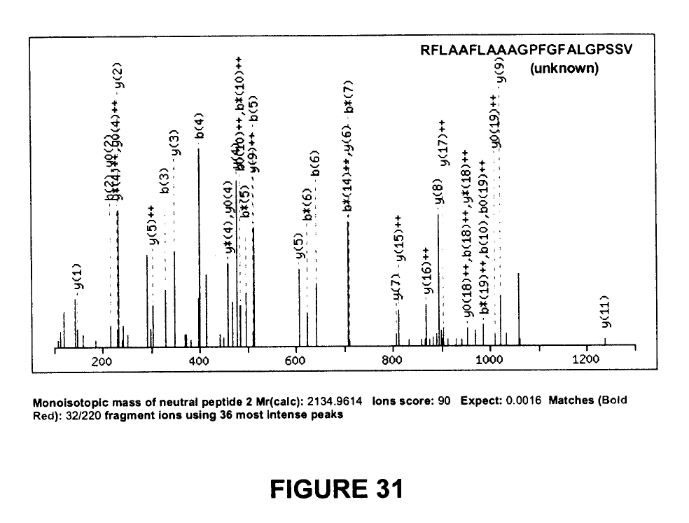

FIGS. 28 and 29 identify Mammalian Scratch as the antigen.

### Discussion

VB3-011, an IgG MAb, was generated from peripheral blood lymphocytes (PBL) isolated from a patient diagnosed with a grade II astrocytoma, using Hybridomics™ and ImmunoMine™ Viventia's proprietary platform technologies (See WO97/044461). The antibody exhibits reactivity to a host of other cell lines each of which is representative of different cancer indications. Despite this demonstration of broad tumor-cell type reactivity, VB3-011 shows limited binding to normal tissue. VB3-011 was shown to react with at least three soluble forms of chondroitin sulfate; two of these (CSB and CSE) have limited tissue distribution. Therefore, most of the antibody reactivity is attributable to CSA and to a lesser extent Hyaluronic acid, thereby identifying CSA or related glycans as key reactive moieties of the antigen molecule. Since CSA molecules are characterized by (1-4) GlcNAc/Glucuronate structures they also resemble the lectin —Neu5Ac (α2→6) Gal(β1→4)Glucuronate, recognized by Hemagglutinin (HA). Hence, a new reagent that would enable lectin-based purification was generated as follows. Recombinant HA was immobilized to anti-HA antibody by coupling with Dimethylpimelimidate (DMP), such that when used as an IP agent, HA recognizes the antigenic epitope on the cell surface. Membrane preparations were affinity purified with immobilized-HA, and the eluates subjected to SDS-PAGE and WB analysis, subsequently probed with VB3-011 antibody.

Western blots of eluates probed with VB3-011 detected a ˜50 kDa protein on 1D-PAGE that further resolved into a ˜36 kDa band on 2D-PAGE analysis. LC-MS/MS analysis of the 1D and 2D spots identified Mammalian Scratch as the antigen with molecular weight 36 kDa (of ˜50 kDa observed by WB analysis of 1D-PAGE), thus attributing the rest to the presence of the glycan, 4-sulfated, Neu5Ac (α2→6) Gal(β1→4)Glucuronate. The detection of a 36 kDa spot on 2D-PAGE matched the molecular weight and isoelectric point [(pl), i.e., 9.7±0.2] characteristic of Mammalian Scratch.

The protein sequences recovered by denovo sequencing from MS/MS fragment ion analyses, resulted in 67% coverage with 16 out of 17 peptides showing 100% homology to the Mammalian Scratch sequence found in the database (gi|13775236). One peptide, RFLAAFLAAAGPFGFALGPSSV, from peptide mass 2134.985448, showed 100% homology in the flanking sequences but not with the sequence in the middle, indicating an identification of a novel sequence. The presence of this sequence is responsible for the only transmembrane domain available on the protein and places Scratch on the cell-surface as opposed to the cytosol. This is the first report depicting Scrt as a cell-surface tumor antigen. Thus, Scratch associated with CSA constitutes the complete antigen for VB3-011.

### Example 4

### Antigen Identification Protocol

First a preliminary characterization step is performed to determine (a) the nature of the epitope (N-/O-glycosylated or a peptide) and (b) whether the antigen is “blottable” or “non-blottable” (whether or not it can be detected by gel-based assays).

### For Blottable Antigens:

1. Preliminary Immunoprecipitation/Western Blot:
(a) Comparison with Isotype-matched controls (to ensure that the binding of antigen to the antibody is specific)
(b) Comparison of expression between positive and negative cell line
2. Stringent IP/Western Blot:
(a) Stringent conditions such as 2×/3× pre-clearing to ensure specificities and establishconditions for antigen purification for each antibody
(b) WB analysis with Isotype-matched control and Mab in study
3. Cell-Panel Screening:
(a) Selection of at least 6 cell lines (4 positive, 2 negative) and IP/WB analysis for each of these using the Mab in study
(b) Consistency in the expression of antigen band in positives and negatives
4. 2D-PAGE
(a) Determining isoelectric point (pl) by 1D and Determination of Mol wt by 2D analysis
(b) Transfer to PVDF membranes and probed with cognate antibody
5. Peptide Extraction for MS
(a) In-gel digestion of 1D bands and 2D spots
(b) Desalting and concentrating with μC18 columns
(c) Reconstitution into MS-compatible buffer
6. MS Analysis:
(a) TOF-MS analysis using nanospray for positive and negative cell lines
(b) Mass reconstruction and PMF analysis
(c) MS/MS fragmentation of abundant peptide ions
(d) De-novo sequencing leading to sequence ID
(e) IDA-static for low-detecting peptides
7. Confirmation of the Antigen
(a) Analyze protein sequence to check if the identified protein is a cell-surface one or a false positive
(b) Compare the pl and Mol.wt vs the amino acid sequence, transmembrane domains etc.
8. Validation
(a) If a known antigen, then commercially available antibodies may be used for IP and WB
9. Epitope ID
(a) Identify potential HLA-binding motifs_ potential epitope sequences
(b) Chemically synthesize these sequences preferably overlapping
(c) Peptide ELISA to detect binding
(d) Competition assay to confirm specificity

### For Non-Blottable Antigens—if Glycosylation Masks the Epitope:

Deglycosylation and IP
(a) If N/O-glycan-associated—remove glycan by N/O-glycanase digestion
(b) Use the deglycosylated membranes for IP and steps 1-9 as mentioned above

### For Non-Blottable Antigens—if Glycan is a Part of the Antigen Complex:

Glycan-Directed Purification
(a) Identify glycan
(b) Identify specific lectin that binds the glycan
(c) Generate an immobilized lectin to specifically purify glycan-associated proteins
(d) Continue Steps 1-9 as above

### For Non-Blottable Antigens Due to Extreme Hydrophobicity.

HTP Antigen ID method:
(a) Immunoprecipitation—pre-fractionation
(b) PF-2D separation
(c) Cap-LC-MS separation and analysis
(d) LC-IDA and MS/MS ion fragmentation

TABLE 1
List of recovered peptides

| | | | | |
|---|---|---|---|---|
| 1998.27 | 3 | 22 | PEDPSETEPAAPRPGASAPR | 12 |
| 1151.24 | 6 | 15 | PSETEPAAPR | 13 |
| 1 | | | | |
| 3140.68 | 26 | 56 | RVFLAAFAAALGPLSFGFALGYSSPAIPSLQR | 14 |
| | | | A | |
| 2916.29 | 64 | 93 | RLDDAAASWFGAVVTLGAAAGGVLGGWLVD | 15 |
| | | | RA | |
| 889.04 | 216 | 223 | RQEAMAALRF | 16 |
| 2984.32 | 224 | 249 | RFLWGSEQGWEDPPIGAEQSFHLALLRQ | 17 |
| 4263.10 | 427 | 463 | KEFSSLMEVLRPYGAFWLASAFCIFSVLFTLF | 18 |
| | | | CVPEIKG | |
| 1401.54 | 466 | 477 | KTLEQITAHFEGR | 19 |
| | 292 | 302 | SLASVVVGVIQ | 20 |

TABLE 2
Table 2: Increase in median fluorescence for VB3-011 over
an isotype-matched control for each cell line used in the studay.

| Cell line | MF* |
|---|---|
| A375 | 11.5 |
| U118MG | 6.1 |
| U87MG | 4.6 |
| MDA-MB- | 4.6 |
| 435S | |
| PANC-1 | 2.1 |
| DAUDI | 1.1 |

TABLE 3

| | | | |
|---|---|---|---|
| | | Peptide | |
| Start | End | mass | Description (Peptide sequence) |

| | | | |
|---|---|---|---|
| 28 | 50 | | A.RFLAAFLAAAGPFGFALGPSSV.Y |
| | | | (SEQ ID NO: 2) |
| 92 | 117 | | R.PELATAAGGYINGDAAVSEGYAADAF.F |
| 4 | 8 | 592.7360 | R.SFLVK.K |
| 12 | 26 | 1601.6900 | K.LDAFSSADLESAYGR.A |
| 62 | 74 | 1345.5360 | K.GPSPEPMYAAAVR.G |
| 75 | 123 | 4719.1140 | R.GELGPAAAGSAPPPTPRPELATAAGGYINGDAAVSEGYAADAFFITDGR.S |
| 128 | 158 | 2457.4690 | K.ASNAGSAAAPSTASAAAPDGDAGGGGGAGGR.S |
| 159 | 167 | 786.8430 | R.SLGSGPGGR.G |
| 172 | 179 | 731.7640 | R.AGAGTEAR.A |
| 180 | 190 | 840.8940 | R.AGPGAAGAGGR.H |
| 199 | 208 | 1099.1660 | K.TYATSSNLSR.H |
| 215 | 222 | 888.9760 | R.SLDSQLAR.R |
| 230 | 247 | 2085.5280 | K.VYVSMPAMAMHLLTHDLR.H |
| 256 | 268 | 1598.8890 | K.AFSRPWLLQGHMR.S |
| 284 | 288 | 578.6260 | K.AFADR.S |
| 293 | 302 | 1157.3120 | R.AHMQTHSAFK.H |
| 312 | 316 | 564.6820 | K.SPALK.S |
| 317 | 321 | 623.7070 | K.SYLNK.H |
| 330 | 348 | 1642.8320 | K.GGAGGPAAPAPPQLSPVQA- |

Table 3: List of peptides along with their respective calculated masses obtained after the reconstruction step is as given in the above table.

While the invention has been described in connection with specific embodiments thereof, it will be understood that it is capable of further modifications and this application is intended to cover any variations, uses, or adaptations of the invention following, in general, the principles of the invention and including such departures from the present disclosures as come within known or customary practice within the art to which the invention pertains and as may be applied to the essential features herein before set forth, and as follows in the scope of the appended claims.

1. A method for identifying a tumor antigen specific for an antibody, the method comprising:
a) providing a sample comprising said antigen;
b) contacting said sample with said antibody and determining a physico-chemical characteristic of said antigen's epitope selected from hydrophobicity, molecular weight, charge, accessibility or affinity;
c) pre-purifying said antigen based on the determined physico-chemical characteristic;
d) separating said pre-purified antigen from other components of said sample; and
e) identifying said antigen using mass spectrometry.

2. The method as claimed in claim 1 wherein said step b) comprises determining a reactivity of said antigen to said antibody in a blotting assay.

3. The method as claimed in claim 1 wherein said step c) of pre-purifying is based on affinity of said epitope for a molecule.

4. The method as claimed in claim 3 wherein said step c) of pre-purifying comprises immunoprecipitating said one or more antigen with said antibody or using affinity chromatography.

5. (canceled)

6. The method as claimed in claim 1 wherein step b) comprises determining a degree of hydrophobicity of said antigen.

7. The method as claimed in claim 1 wherein step b) comprises determining a degree of accessibility of said epitope to said antibody.

8. The method as claimed in claim 1 wherein said antigen is a protein or fragment thereof.

9. The method as claimed in claim 8 wherein said protein or fragment thereof is a glycoprotein.

10. The method as claimed in claim 9 wherein said step of pre-purifying c) is a lectin-based affinity chromatography.

11. The method as claimed in claim 3 wherein said step of separating d) is 2D chromatography.

12. The method as claimed in claim 11 wherein said 2D chromatography comprises chromatofocussing in a first dimension and hydrophobicity-based chromatography in a second dimension.

13. The method as claimed in claim 12 wherein said hydrophobicity-based chromatography is reverse phase chromatography.

14. The method as claimed in claim 13 wherein said reverse phase chromatography is non-porous reverse phase chromatography.

15. The method as claimed in claim 8 wherein said pre-purified proteins or fragments thereof are subjected to enzymatic digestion to generate peptides prior to 2D chromatography or electrophoresis, after first dimension (1-D) chromatography or electrophoresis or after 2D chromatography or electrophoresis.

16. The method as claimed in claim 15 wherein said peptides are further separated using porous reverse phase chromatography (porous RPC) after said 2D chromatography.

17. The method as claimed in claim 16 wherein said peptides are injected in said porous RPC such as to preserve elution profile information from previous separation steps.

18. The method as claimed in claim 17 wherein said peptides are injected in said porous RPC using a capillary LC microautosampler for high throughput analysis.

19. The method as claimed in claim 1 wherein said mass spectrometry analysis is selected from Liquid Chromatography-mass spectrometry (LC-MS), nano-electrospray ionization tandem mass spectrometry (ESI-MS/MS) and tandem mass spectrometry (MS/MS).

20. The method as claimed in claim 19 wherein said mass spectrometry analysis comprises comparing mass spectrometry data with protein/peptide databases.

21. The method as claimed in claim 20 wherein said mass spectrometry analysis comprises obtaining a molecular weight for said protein.

22. The method as claimed in claim 1 wherein said step of pre-purifying c) comprises isolating sub-cellular structure/organelle comprising said antigen.

23. The method as claimed in claim 22 wherein said sub-cellular structure is a membrane and said protein is a membrane protein.

24. The method as claimed in claim 23 wherein said membrane protein is expressed on cells of interest and wherein membrane proteins from a sample comprising cells of interest and from a sample comprising reference cells are compared to identify cell-specific antigen.

25. The method as claimed in claim 24 wherein said cells of interest are cancer cells.

26. The method as claimed in claim 1 wherein said antigen is deglycosylated.

27. A system for identifying an antigen comprising:
a) pre-purification means for providing a fraction enriched in said antigen;
b) separating means for separating said antigen from other components in said fraction; and
c) an analysis means for identifying said antigen.

28. The system as claimed in claim 27 further comprising screening means for detecting a presence of said antigen in a sample.

29. The system as claimed in claim 27 wherein said separating means is a 2D separating means.

30. The system as claimed in claim 27 further comprising:
a) a first collecting device for receiving pre-purified antigen fraction; and
b) a second collecting device for receiving separated fractions from said separating means.

31. The system as claimed in claim 30 wherein said second collecting device comprises parts for collecting from a first and a second dimension separation of said 2D separating means.

32. The system as claimed in claim 27 further comprising injecting means for injecting collected fractions in said separating means or said analysis means.

33. The system as claimed in claim 32 further comprising connecting means for operationally connecting said collecting and said injecting means.

34. The system as claimed in claim 27 wherein said analysis means comprises processor means for analyzing data from said analysis means to identify said antigen.

35. The system as claimed in claim 27 wherein said antigen is a protein.

36. The system as claimed in claim 35 wherein said separating means is 2D chromatography and wherein said analysis means is a mass spectrometer.

37. The system as claimed in claim 36 further comprising a chromatographic device to further separate said fractions collected from said separating means prior to analysis by said analysis means.

38. The system as claimed in claim 27 which is substantially automated.
